(19) United States
(12) Patent Application Publication
(10) Pub. No.: US 2026/0160764 A1
(43) Pub. Date: Jun. 11, 2026

(54) TWENTY-FOUR COLOR MOUSE IMMUNOPROFILING PANEL AND REAGENT KIT FOR SPECTRAL FLOW CYTOMETERS

(71) Applicant: Cytek Biosciences, Inc., Fremont, CA (US)

(72) Inventor: Mary Beth Hanley, Fremont, CA (US)

(21) Appl. No.: 19/362,392

(22) Filed: Oct. 18, 2025

Related U.S. Application Data
(60) Provisional application No. 63/709,022, filed on Oct. 18, 2024.

(51) Int. Cl. G01N 33/569; G01N 33/68

(52) U.S. Cl. CPC G01N 33/56972; G01N 33/6872

(57) ABSTRACT

In one embodiment, a method of forming a twenty-four (24) color flow cytometry panel is disclosed for use with a full spectrum or spectral flow cytometer with at least three excitation lasers. A reagent kit for mouse cell immunoprofiling associated with the twenty-four (24) color flow cytometry panel is disclosed. The reagent kit is comprised of optimally selected fluorochrome conjugated monoclonal antibodies for enumerating cell lineages of mouse cells. The fluorescence flow cytometry based mouse kit enumerates a more complete set of mouse cell lineage markers in a single tube.

### CROSS-REFERENCE TO RELATED APPLICATIONS

This patent application is a non-provisional application claiming the benefit of United States (US) Provisional Patent Application No. 63/709,022 titled TWENTY-FOUR COLOR MOUSE IMMUNOPROFILING PANEL AND REAGENT KIT FOR FLOW CYTOMETERS filed on Oct. 18, 2024 by inventor Mary Hanley.

This patent application is related to United States (US) Non-Provisional patent application Ser. No. 17/304,843 titled METHODS OF FORMING MULTI-COLOR FLUORESCENCE-BASED FLOW CYTOMETRY PANEL filed on Jun. 26, 2021 by inventors Maria Jaimes et al., incorporated herein by reference for all intents and purposes. (US) Non-Provisional patent application Ser. No. 17/304,843 claims the benefit of United States (US) Provisional Patent Application No. 63/045,040 titled METHODS OF FORMING MULTI-COLOR FLUORESCENCE-BASED FLOW CYTOMETRY PANEL filed on Jun. 26, 2020 by inventors Maria Jaimes et al., incorporated herein by reference for all intents and purposes. (US) Non-Provisional patent application Ser. No. 17/304,843 also claims the benefit of United States (US) Provisional Patent Application No. 63/045,103 titled METHODS OF FORMING MULTI-COLOR FLUORESCENCE-BASED FLOW CYTOMETRY PANEL filed on Jun. 27, 2020 by inventors Maria Jaimes et al., incorporated herein by reference for all intents and purposes.

This patent application is further related to United States (US) patent application Ser. No. 15/659,610 titled COMPACT DETECTION MODULE FOR FLOW CYTOMETERS filed on Jul. 25, 2017 by inventors Ming Yan et al., incorporated herein by reference for all intents and purposes. This patent application is further related to U.S. patent application Ser. No. 15/498,397 titled COMPACT MULTI-COLOR FLOW CYTOMETER filed on Apr. 26, 2017 by David Vrane et al. that describes a flow cytometer with which the embodiments can be used and is incorporated herein by reference for all intents and purposes. This patent application is further related to U.S. patent application Ser. No. 16/418,942 titled FAST RECOMPENSATION OF FLOW CYTOMETERY DATA FOR SPILLOVER READJUSTMENTS filed on May 21, 2019 by Zhenyu Zhang that describes matrices with which the embodiments can be used and is incorporated herein by reference for all intents and purposes.

### FIELD

The embodiments of the invention relate generally to fluorochrome and marker selection to analyze biological samples with a flow cytometer.

### BACKGROUND

Flow cytometry is a technology that provides rapid analysis of physical and chemical characteristics of single cells in solution. Flow cytometers utilize lasers as light sources to produce both scattered and fluorescent light signals that are read by detectors such as photodiodes or photomultiplier tubes. Cell populations can be analyzed and/or purified based on their fluorescent or light scattering characteristics. Flow cytometry provides a method to identify cells in solution and is most commonly used for evaluating peripheral blood, bone marrow, and other body fluids.

Flow cytometry is generally used in the analysis of biological cells. Examples of biological cells include Astrocyte, Basophil, B Cell, Embryonic Stem Cell, Endothelial Cell, Eosinophil, Epithelial Cell, Erythrocyte, Fibroblast, Hematopoietic Stem Cell, Macrophage, Mast Cell, Myeloid-derived suppressor cells (MDSCs), Megakarocyte, Mesenchymal Stem Cell, Microglia, Monocyte, Myeloid Dendritic Cell, Naïve T Cell, Neurons, Neutrophil, NK Cell, Plasmacytoid Dendritic Cell, Platelets, Stromal Cells, T Follicular Helper, Th1, Th2, Th9, Th17, Th22, and Treg. Although flow cytometry was developed originally for analysis of relatively large mammalian cells, it is finding increased use by microbiologists.

The basic principle of flow cytometry is the passage of cells in single file in front of a laser so they can be detected, counted and sorted. A beam of laser light is directed at a hydrodynamically-focused stream of fluid that carries the cells. Several detectors are carefully placed around the stream, at the point where the fluid passes through the light beam. The stream of fluid is focused so that the cells pass through the laser light one at a time.

In hydrodynamic focusing, the sample fluid is enclosed by an outer sheath fluid and injected through a nozzle or cuvette. The nozzle or cuvette can be cone shaped causing a narrowing of the sheath and subsequent increase in the fluid velocity. The sample is introduced into the center and is focused by the Bernoulli effect. This allows the creation of a stream of particles in single file. Under optimal conditions (laminar flow) there is no mixing of the central fluid stream and the sheath fluid.

Once the cells are lined up in a single file flow, they are passed through one or more lasers. One or more detectors are placed proximate the point where the fluid passes the laser beam. Those detector(s) in line with the light beam, and typically up to 20 degrees offset from the laser beam's axis, are used to measure Forward Scatter or FSC. This FSC measurement can give an estimation of a particle's size with larger particles refracting more light than smaller particles, but this can depend on several factors such as the sample, the wavelength of the laser, the collection angle and the refractive index of the sample and sheath fluid.

Other detector(s) are placed perpendicular to the beam and are used to measure Side Scatter (SSC). The SSC can provide information about the relative complexity of internal structures or surface structures (i.e. granularity) of a cell or particle; however as with forward scatter this can depend on various factors.

Both FSC and SSC are unique for every particle and a combination of the two may be used to roughly differentiate cell types in a heterogeneous population such as blood. However, this depends on the sample type and the quality of sample preparation, so fluorescent labeling is generally required to obtain more detailed information.

In modern flow cytometry, cells are fluorescently labelled and then excited by laser(s) to emit light at varying wavelengths. The fluorescence can then be measured to determine the amount and type of cells present in a sample. In preparation for flow cytometric analysis, single cells in suspension are fluorescently labeled, typically with a fluorescently conjugated monoclonal antibody. Antibodies are stained with a fluorophore (fluorochrome or dye) and introduced to the cell population, where they bind to cell markers.

Fluorophores are fluorescent markers used to detect the expression of cellular molecules such as proteins or nucleic acids. They accept light energy (for example, from a laser) at a given wavelength and re-emit it at a longer wavelength. These two processes are called excitation and emission. Emission follows excitation extremely rapidly, commonly in nanoseconds and is known as fluorescence.

When a fluorophore absorbs light, its electrons become excited and move from a resting state, to a maximal energy level called the excited electronic singlet state. The amount of energy required for this transition will differ for each fluorophore. The duration of the excited state depends on the fluorophore and typically lasts for 1-10 nanoseconds. The fluorophore then undergoes a conformational change, the electrons fall to a lower, more stable energy level called the electronic singlet state, and some of the absorbed energy is released as heat. The electrons subsequently fall back to their resting state releasing the remaining energy as fluorescence.

Cells express characteristic (proteins, lipids, glycosylation, etc.) that can be used to help distinguish unique cell types. These markers are referred to as cell markers that can be expressed both extracellularly on the cells surface (surface or extracellular cell marker) or as an intracellular molecule (intracellular cell marker). Markers are generally functional membrane proteins involved in cell communication, adhesion, or metabolism. Surface and intracellular cell markers can be used for a variety of cell types including immune cells, stem cells, central nervous system cells, and more.

Antibodies can specifically bind to cell markers. The affinity between the paratope region of antibodies and the corresponding epitope region of cell markers are a very useful way to label and identify a specific cell population. However, the cell markers will often be expressed on more than one cell type. Therefore, flow cytometry staining strategies have led to methods for immunophenotyping cells with two or more antibodies simultaneously.

CD markers (cluster of differentiation markers) are used for the identification and characterization of leukocytes and the different subpopulations of leukocytes. Many immunological cell markers are CD markers and these are commonly used for detection in flow cytometry of specific immune cell populations and subpopulations. The majority of flow cytometer analysis are conducted on leukocytes; however, the general principle of the invention is applicable to other bodily fluids.

Antigens and cell markers are often used interchangeably, as many antigens function as cell markers. In flow cytometry, antigens are classified as primary, secondary, and tertiary markers based on their expression levels, continuity, and role in defining cell populations. Primary markers have high, clearly distinguishable expression used for initial cell population identification, while secondary markers show continuous expression and are used for sub-setting and further differentiation. Tertiary markers are often the most critical, expressed at low levels, but are crucial for answering specific experimental questions. Primary markers with the highest expressing antigens are generally assigned to the dimmest fluorochromes.

The fluorescently labelled cell components are excited by the laser and emit light at a longer wavelength than the light source. The detectors therefore pick up a combination of scattered and fluorescent light. The intensity of the emitted light is directly proportional to the antigen density or the characteristics of the cell being measured. Data from the detectors can then be analyzed by a computer using special software. The computer can be coupled in communication with the flow cytometer to hasten data analysis.

Fluorescence measurements taken at different wavelengths can provide quantitative and qualitative data about fluorophore-labeled cell surface receptors or intracellular molecules such as DNA and cytokines. Most flow cytometers use separate channels and detectors to detect emitted light, the number of which vary according to the instrument and the manufacturer.

The need to understand the mechanisms and pathways of immune evasion seen either post immunotherapy or during natural immune responses to cancer, autoimmunity, and infectious diseases, requires methods and protocols which will enable a deeper profiling of the immune system. Greater characterization of immune subpopulations allows for more informed decisions regarding the identification of targetable biomarkers and the development of new therapeutic approaches. Unraveling the complexity of the human immune response requires the ability to perform high throughput, in-depth analysis, at the single cell and population levels.

Sample availability can often be limited, especially in cases of clinical trial material, when multiple types of testing are required from a single sample or timepoint. Maximizing the amount of information that can be obtained from a single sample not only provides more in-depth characterization of the immune system, but also serves to address the issue of limited sample availability.

For research blood from C5BL/6 mice are often used. C57BL/6 mice are one of the most popular strains used in research because of their genetic purity. C57BL/6 mouse colonies are genetically identical within each strain, making them free of genetic differences that could impact research results. Splenocytes, extracted from mice spleens, can be used for a wide variety of immunology-based applications.

### BRIEF SUMMARY

The embodiments of the invention are summarized by the claims that follow below. In some aspects, the techniques described herein relate to a twenty-four (24) color mouse immunoprofiling panel using a spectral flow cytometer, the panel including:

VIOLET

| SPECIFICITY/MARKER | ANTIBODY/CLONES | FLUOROCHROME |
|---|---|---|
| XCR1 | ZET | cFluor V435 |
| CD11c | N418 | cFluor V450 |
| MertK | DS5MMER | BV480 |
| NK1.1 | PK136 | cFluor V505 |
| CD45 | 30-F11 | cFluor V547 |
| CD8 | 53-6.7 | cFluor V605 |
| B220/CD45R | RA3-6B2 | cFluor V670 |
| Siglec F (CD170) | 1RNM44N | BV711 |
| CD4 | RM4-5 | cFluor V780 |

BLUE

| SPECIFICITY/MARKER | ANTIBODY/CLONES | FLUOROCHROME |
|---|---|---|
| CD3 | 17A2 | cFluor B515 |
| CD19 | 1D3 | cFluor B532 |
| Ly-6G | 1A8 | cFluor B548 |
| CD64 | X54-5/7.1 | cFluor BYG575 |
| F4/80 | BM8.1 | cFluor BYG610 |
| TCR beta | H57-597 | cFluor BYG645 |
| CD317 | 927 | cFluor BYG667 |
| CD49b | HMa2 | cFluor BYG710 |
| I-A/I-E | M5/114.15.2 | cFluor BYG750 |
| CD62L | MEL-14 | cFluor BYG781 |

RED

| SPECIFICITY/MARKER | ANTIBODY/CLONES | FLUOROCHROME |
|---|---|---|
| CD25 | PC61.5 | cFluor R659 |
| CD11b | M1/70 | cFluor R685 |
| CD44 | IM7 | cFluor R720 |
| | | ViaDye Red |
| TCR g/d | GL3 | cFluor R780 |
| Ly-6C | HK1.4 | cFluor R840 |

In some aspects, the techniques described herein relate to a method 1; selecting twenty-four (24) clones specific to the twenty-four (24) cell markers; identifying twenty-four (24) fluorochromes, to be used in the twenty-four (24) color flow cytometry panel; conjugating the twenty-four (24) clones with the twenty-four (24) fluorochromes to form twenty-four (24) fluorochrome conjugated antibodies; calibrating at least three (3) lasers and at least twenty-five (25) photodetectors in the spectral flow cytometer; comparing resolution of each fluorochrome in a multicolor single tube sample versus a twenty-four (24) combined control sample; staining the biological cells of interest with the twenty-four (24) fluorochrome conjugated antibodies, including the twenty-four (24) fluorochromes and twenty-four (24) clones specific to the twenty-four (24) cell markers, to create a multicolor single tube sample; analyzing the multicolor single tube sample through the spectral flow cytometer including the at least twenty-five (25) photodetectors; receiving data from the at least twenty-five (25) photodetectors of the spectral flow cytometer; and processing the received data using a computer processor to analyze the multicolor single tube sample of the biological cells of interest.

In some aspects, the techniques described herein relate to a method, wherein pairing the twenty-four (24) fluorochromes with the twenty-four (24) selected cell markers includes; assigning a dimmest fluorochrome to a highest expressing antigen; assigning tertiary markers to bright fluorochromes; and avoiding placing highly expressed antigens adjacent to co-expressed antigens with lower expression for fluorochromes with a same primary excitation laser or similar emission wavelengths.

In some aspects, the techniques described herein relate to a method, wherein the biological cells of interest include splenocyte and bone marrow cells.

In some aspects, the techniques described herein relate to a method, wherein selecting the twenty-four (24) fluorochromes includes, quantifying uniqueness of each of the twenty-four (24) fluorochromes.

In some aspects, the techniques described herein relate to a method, wherein selecting the twenty-four (24) fluorochromes includes, analyzing spectra of each of the twenty-four (24) fluorochromes using the spectral flow cytometer.

In some aspects, the techniques described herein relate to a method, wherein selecting the twenty-four (24) fluorochromes includes, comparing the spectra of a pairing of twenty-four (24) or fluorochromes; and assigning a similarity index to each pairing of fluorochromes.

In some aspects, the techniques described herein relate to a method, wherein selecting the twenty-four (24) fluorochromes further includes, determining a threshold similarity index value and not selecting at least one fluorochrome of the pair of fluorochromes with a similarity index value higher than the threshold similarity index value.

In some aspects, the techniques described herein relate to a method, wherein selecting the twenty-four (24) fluorochromes includes, choosing the twenty-four (24) fluorochromes with a lowest similarity index value.

In some aspects, the techniques described herein relate to a method, wherein a range for the lowest similarity index value that will produce high resolution data is between 0.95 and 1.0.

In some aspects, the techniques described herein relate to a method, wherein identifying the twenty-four (24) fluorochromes includes: determining a complexity index for the twenty-four (24) fluorochromes; and determining a threshold complexity index above which the twenty-four (24) fluorochromes are not considered optimal.

In some aspects, the techniques described herein relate to a method, wherein a range of the threshold complexity index is between fifty-two (52) to fifty-six (56).

In some aspects, the techniques described herein relate to a reagent kit for mouse cell immunoprofiling by a spectral flow cytometer having at least three (3) lasers, the reagent kit including: a plurality 1.

In some aspects, the techniques described herein relate to a reagent kit, wherein the at least three (3) lasers are violet, red, and blue lasers.

In some aspects, the techniques described herein relate to a method of gating a twenty-four (24) color mouse cell immunoprofiling panel, the method including; sequentially gating for total live splenocytes by red blood cell exclusion, singlets, and viable CD45+ events; excluding debris from the viable CD45+ events; gating for NK cells and CD3+ NKT-like cells by expression of CD49b versus NK1.1, and subsequently gated on CD3 versus B220; identifying basophils (CD49b+CD45dim) after excluding the 49b+NK1.1+; determining plasmacytoid dendritic cells (PDCs, CD317++B220+) after excluding non-basophils; identifying B cells in non-PDC cells by positive expression of CD19 when gated versus B220; identifying γδT cells by co-expression of CD3 and TCRγδ from B220−CD19− gated event; gating for T cells on a CD3 versus TCRSYMBOL plot (gating on CD3+TCRSYMBOL+), from the non-γδ-T events; distinguishing CD4+ and CD8+ T cell subsets from the total T cell gate (CD4+CD8− and CD8+CD4− events) and then further subdivided into naïve and memory subsets by expression of CD62L and CD44 for each T cell subset (CD4+ and CD8+); identifying CD4+CD25+ T cells from a CD4 versus CD25 plot; and gating for dendritic cells (DCs) on a CD11c versus I-A/I-E plot from non-T cell events and DCs are further subdivided into cDC1 and cDC2 populations using CD8 vs. XCR1 and CD4 vs. CD11b plot.

### BRIEF DESCRIPTIONS OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1A is a basic conceptual diagram of a flow cytometer system.

FIG. 1B is a conceptual diagram of a fluorochrome, an antibody, and a cell.

FIG. 1C is a conceptual diagram of forming a reference sample with a bead.

FIG. 2A is an overall flowchart of a method for performing an experiment with a biological sample and/or running calibration beads through a flow cytometer.

FIG. 2B is a diagram of a calibrating process of a flow cytometer with single stained compensation controls to generate an initial spillover matrix or reference matrix with levels of compensation.

FIG. 2C is a diagram of running a sample through the flow cytometer resulting in a mixed sample event vector with an overlapping spectral profile due to multi-stained cells or particles.

FIG. 2D is a diagram of a processing using an inverse matrix (determined from the initial spillover matrix and/or the initial reference matrix with fine adjustments) on the event data to generate a compensated sample event vector or an unmixed sample event vector.

FIG. 2E1-2E2 is a schematic diagram of a full spectrum or spectral flow cytometer.

In FIG. 2F1-2F2, the configuration details of the photo detectors in the detector modules for a full spectrum or spectral flow cytometer is shown.

FIG. 2G1-2G2 illustrates the individual spectrum signature of each color laser and combined full spectrum or spectral signature of an exemplary fluorochrome.

FIG. 3 is a listing of the exemplary cell markers and fluorochromes in a 28 color Optimized Multicolor Immunofluorescence Panel (OMIP).

FIG. 4A illustrates an exemplary 24-Color Mouse Immunoprofiling Panel.

FIG. 4B illustrates a listing of the cell markers in the exemplary 24-color mouse immunoprofiling panel paired with the subset population identified.

FIG. 5 (5A to 5C) illustrate dot plots of an exemplary cell gating strategy for identifying mouse cells using the 24-color mouse immunoprofiling panel of FIG. 4A.

FIG. 6 illustrates an exemplary Mouse Kit Panel Hierarchy of the 24-Color Mouse Immunoprofiling Panel.

FIG. 7A illustrates an exemplary 24-color mouse immunoprofiling panel with recommended control type for each marker/fluorochrome pair when the sample is extracted from Splenocytes.

FIG. 7B illustrates an exemplary 24-color mouse immunoprofiling panel with recommended control type for each marker/fluorochrome pair when the sample is extracted from Bone Marrow.

FIGS. 8 (8-1 to 8-3) illustrates a similarity matrix with similarity indexes and a computation of a complexity index for forty fluorochrome sample and a full spectrum or spectral flow cytometer having five lasers and five detector arrays such as shown in FIG. 2E.

FIG. 9 introduces a simple 3 detector and two fluorochrome example to show and describe how the similarity index for a pair of fluorochromes and the complexity index for a set of two fluorochromes are generated.

FIG. 10 illustrates two reference control vectors for two reference samples of two fluorochromes in continuing with the example introduced by FIG. 9.

FIG. 11 illustrates a simple spillover matrix for the example introduced by FIG. 9.

FIG. 12 illustrates event vectors obtained by running a mixed sample through a flow cytometer for the two reference samples and two fluorochromes introduced by FIG. 9.

FIG. 13 illustrates a spectra signature obtained by a more complex flow cytometer with 64 detectors that generates a 64-dimension vector representing that spectral signature to contrast it with the simplified example.

FIG. 14 illustrates a simple example of a similarity index and its association with the reference control vectors of two reference samples.

FIGS. 15-16 introduces the matrices and linear algebra that can be used to compute a complexity index.

FIG. 17 illustrates three simple complexity examples with a set of two fluorochromes.

FIGS. 18 (18-1 to 18-4) illustrates a similarity matrix with similarity indexes and example computations of a complexity index for a 35 fluorochrome sample.

FIG. 19 is a chart illustrating a classification of antigens/cell markers that can affect the detected data.

FIG. 20A-20B are block diagrams of a computer system that can execute software instructions to display a graphical user interface and remotely interact with a web-based spectrum viewer software application.

FIG. 21 is a top view of an optical plate assembly in a modular flow cytometry system with three excitation lasers.

FIG. 22 is a top view of an optical plate assembly in a modular flow cytometry system with five excitation lasers, including an ultraviolet (UV) excitation laser, of the full spectrum or spectral flow cytometer.

FIG. 23 illustrates an exemplary 24-color mouse immunoprofiling reagent kit.

### DETAILED DESCRIPTION

In the following detailed description of the embodiments of the invention, numerous specific details are set forth in order to provide a thorough understanding of the present invention. However, it will be obvious to one skilled in the art that the embodiments of the invention may be practiced without these specific details. In other instances, well known methods, procedures, components, and circuits have not been described in detail so as not to unnecessarily obscure aspects of the embodiments of the invention.

The embodiments include a method, apparatus and system for a multi-color fluorescence-based flow cytometry panel for a mouse immunoprofiling assay and reagent kit and method of using the mouse immunoprofiling panel and kit.

Full spectrum or spectral flow cytometry is a technology that enables the development of such highly multiparametric panels. A full spectrum flow cytometer measures the entire fluorochrome emission, from ultra-violet to near infra-red, across multiple lasers using many more detectors compared to a conventional flow cytometer. A spectral flow cytometer can measure a subset of the full spectrum over a continuous light spectrum of wavelengths. Both the full spectrum and the spectral flow cytometer can produce very specific spectral fingerprints that are used to mathematically distinguish one fluorophore from another, even when their maximum emissions (the primary component measured by a conventional flow cytometer) are very similar. Leveraging this full spectrum or spectral technology, the ability to combine 30 or more fluorescently labeled antibodies becomes possible using a fluorescence-based flow cytometer.

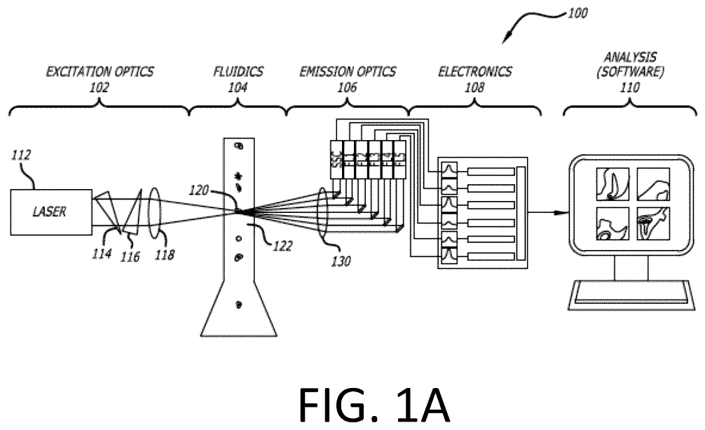

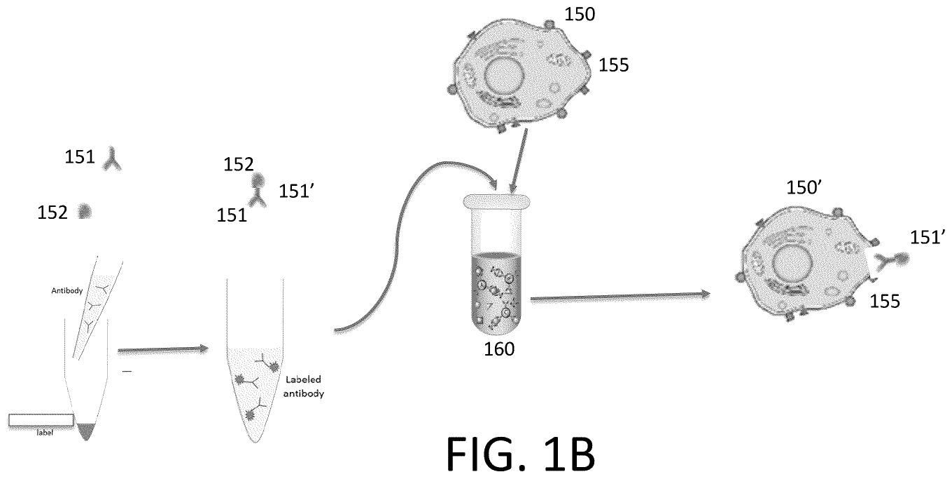

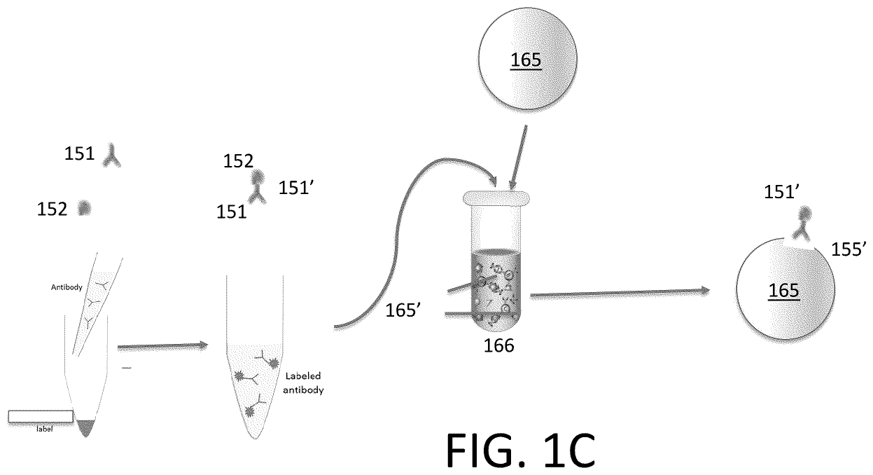

Referring now to FIG. 1A, a basic conceptual diagram of a flow cytometer system 100 is shown. Various embodiments of the flow cytometer 100 may be commercially available. Five major subsystems of the flow cytometer system 100 include an excitation optics system 102, a fluidics system 104, an emission optics system 106, an acquisition system 108, and an analysis system 110. Generally, a “system” includes hardware devices, software devices, or a combination thereof.

The excitation optics system 102 includes, for example, a laser device 112, an optical element 114, an optical element 116, and an optical element, 118. Example optical elements include an optical prism and an optical lens. The excitation optics system 102 illuminates an optical interrogation region 120. The fluidics system 104 carries fluid samples 122 through the optical interrogation region 120. The emission optics system 106 includes, for example, an optical element 130 and optical detectors SSC, FL1, FL2, FL3, FL4, and FL5. The emission optics system 106 gathers photons emitted or scattered from passing particles. The emission optics system 106 focuses these photons onto the optical detectors SSC, FL1, FL2, FL3, FL4, and FL5. Optical detector SSC is a side scatter channel. Optical detectors FL1, FL2, FL3, FL4, and FL5 are fluorescent detectors may include band-pass, or long-pass, filters to detect a particular fluorescence wavelength. Each optical detector converts photons into electrical pulses and sends the electrical pulses to the acquisition system 108. The acquisition system 108 processes and prepares these signals for analysis in the analysis system 110.

The analysis system 110 can store digital representations of the signals for analysis after completion of acquisition. The analysis system 110 is a computer with a processor, memory, and one or more storage devices that can store and execute analysis software to obtain laboratory results of biological samples (or other types of samples, e.g., chemical) that are analyzed. The analysis system 110 can be further used to calibrate the flow cytometer with compensation controls when initialized, before running a reference sample through the flow cytometer. Reference samples can be formed in different ways to determine spillover vectors for a fluorescent dye or fluorochrome. A fluorochrome can be conjugated with an antibody and then attached to a biological cell or attached to a bead or particle.

Referring now to FIG. 1B, a cell 150, an antibody 151, and a fluorochrome (dye) 152 are coupled together to form a reference sample with direct marking or staining of a cell. The cell 150 has one or more cell marker 155 sites to which an antibody can attach. The fluorochrome (dye) 152 is conjugated with the antibody 151 in advance to form a conjugated antibody 151′. For a reference sample, a single fluorochrome (dye) 152 is conjugated with a single antibody to generate a spillover vector. Subsequently, when analyzing a biological fluid with different unknown counts of cells in the biological fluid, multiple conjugated antibodies with different antibodies and different fluorochrome, can be used and add into the same biological sample.

The conjugated antibodies 151′ and the cells 150 are mixed together in a test tube 160 so the conjugated antibodies 151′ can attached to the desired cell marker sites 155 for the given type of cells 150 to form marked or stained cells 150′ in the sample biological fluid. When run through the flow cytometer, the fluorochromes can be excited by laser light to fluoresce so that the fluorescence can be detected by detectors as events generating an event vector. The event vector can be used to generate a spill over matrix for the fluorochrome. When running a sample biological fluid with unknown counts, the cells counted by a flow cytometer by analyzing the events.

Referring now to FIG. 1C, a conceptual diagram of forming a reference sample with a bead 165 is shown. A bead 165, an antibody 151, and a fluorochrome (dye) 152 are coupled together to form a reference sample with a bead. The bead 165 may have one or more cell marker 155′ sites to which an antibody can attach. As with the cell, the fluorochrome (dye) 152 is conjugated with the antibody 151 in advance to form a conjugated antibody 151′. For a reference sample, a single fluorochrome (dye) 152 is conjugated with a single antibody to generate a spillover vector.

The conjugated antibodies 151′ and the beads 165 are mixed together in a test tube 166 so the conjugated antibodies 151′ can attached to the desired marker sites 155′ for the beads 165 to form marked beads 165′ in a reference sample. When run through the flow cytometer, the fluorochromes can be excited by laser light to fluoresce so that the fluorescence can be detected by detectors as events generating an event vector. The event vector can be used to generate a spill over matrix for the fluorochrome. In this manner, either cells or beads can be used to test and fluorochrome for suitability to be used with a flow cytometer.

### Reference Sample Run

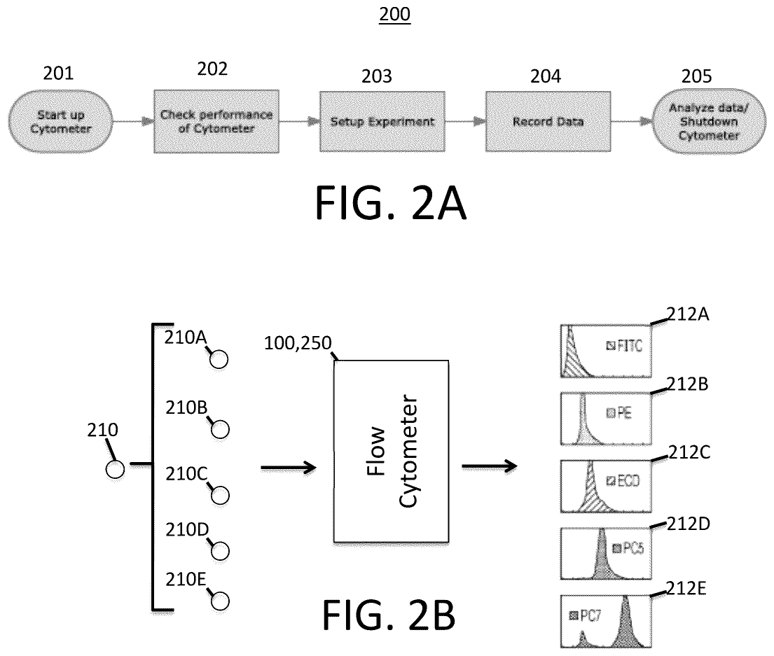

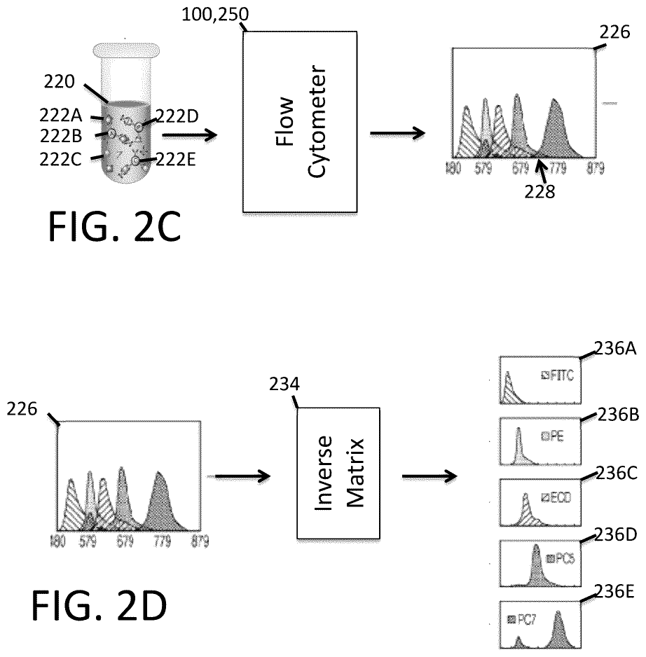

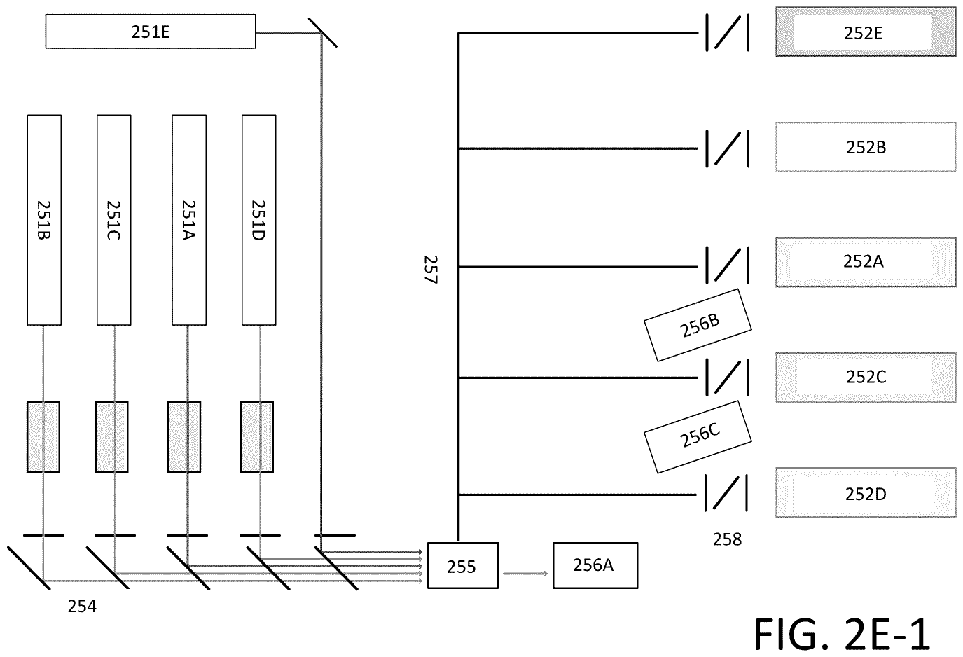

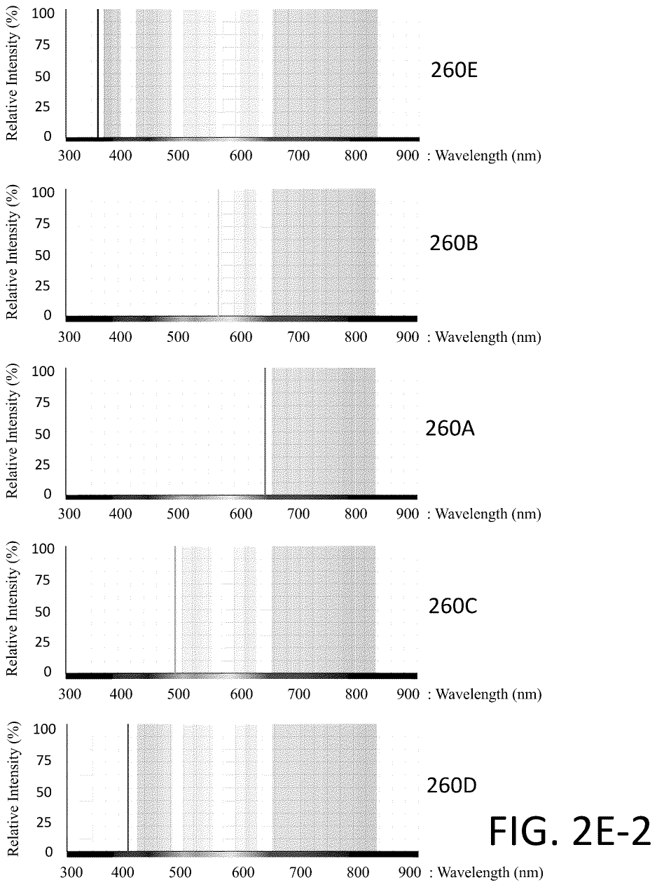

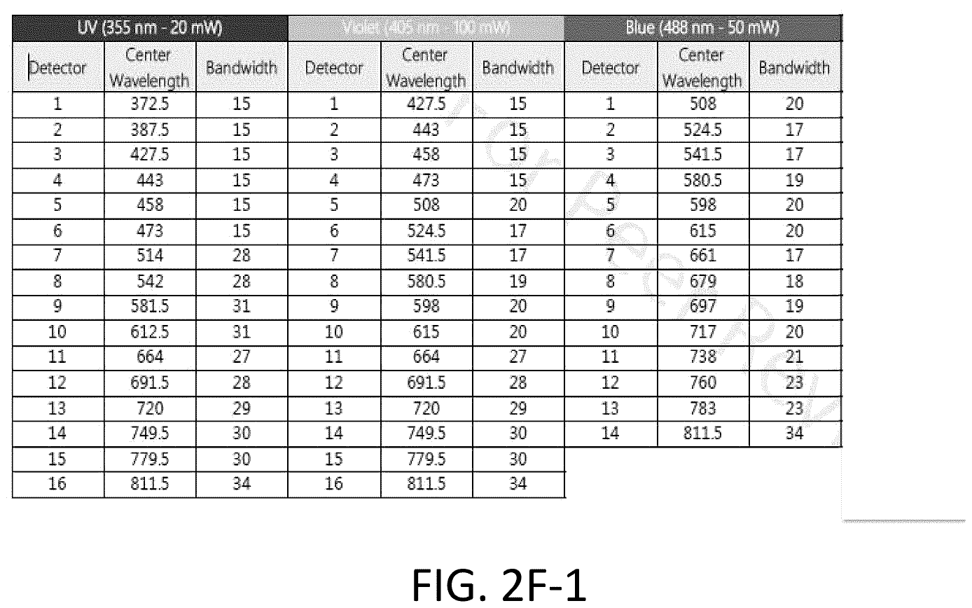

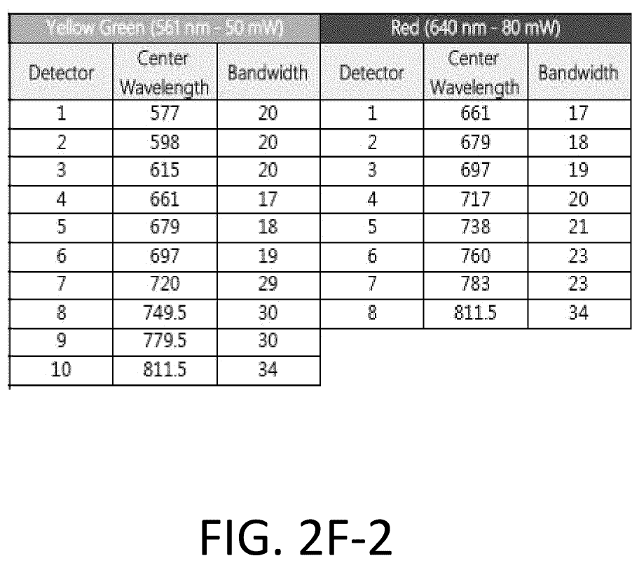

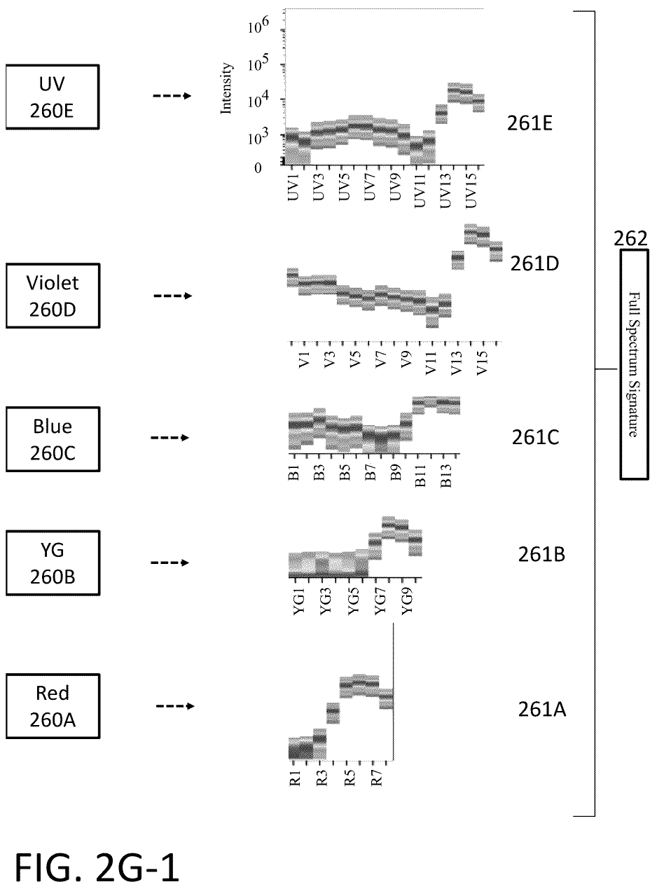

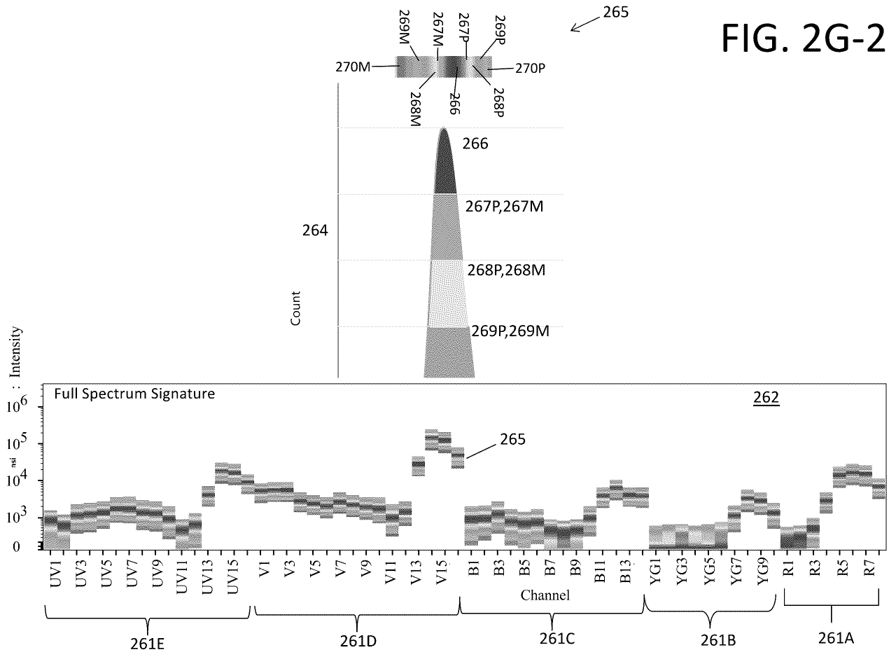

Referring now to FIG. 2A, a flowchart of a method 200 for a flow cytometer is shown. The flow cytometry system 100 of FIG. 1A, or other flow cytometer systems (e.g., system 250 shown if FIG. 2E) disclosed herein, can carry out the method 200. Flow cytometry allows for data collection and analysis of data on single cells or particles of a plurality that are in a sample fluid.

In step 201, the system starts up the flow cytometer. In step 202, the system checks the performance of the flow cytometer and performs calibration if and as needed with calibration beads. If the flow cytometer was recently calibrated (e.g., same day or same hour), this step can be skipped.

In step 203, multiple experiments are setup to run to generate spillover vectors for each dye. A reference sample is prepared (fluorochrome conjugated to an antibody that is attached to a cell or a bead) to initially run to generate event vectors that can be converted into a spillover vector.

In step 204, the reference sample fluid with one fluorochrome is run through the flow cytometer for analysis with the data captured from N detectors being recorded. Multiple runs through the flow cytometer with the same reference sample fluid may be performed to be sure measurements are well understood. The data from N detectors is recorded for each run of the reference sample through the flow cytometer.

In step 205, after the sample fluid or calibration beads are run through the flow cytometer, the recorded data can be analyzed to determine results from the analysis by the flow cytometer.

Each spillover vector for one fluorochrome can be subsequently compared with another spillover vector for another fluorochrome to determine how different combinations of pairs of fluorochromes (dyes) and markers interact and spectrally interfere. The spillover vectors for each dye can be subsequently combined together into a spillover matrix for a total number and types of dye being used together to identify cells/particles in a single sample. Combinations of pairs of spillover vectors (columns) in the spillover matrix can be compared together to determine a similarity index between the two fluorochromes. For each reference sample, the light intensity density for each channel can saved as a reference vector and the data can be binned and plotted to form a full spectrum signature for the given fluorochrome.

The flow cytometer can also be shut down if no further samples or calibration beads are to be run. Alternatively, another sample or more calibration beads can be run through the flow cytometer to obtain and record (save) data and subsequently analyze the recorded data.

In step 205, the system performs single stained compensation controls to generate an initial spillover matrix or reference matrix. When performing multicolor flow cytometry, the system uses single stained samples (reference samples) 210A-210E (collectively referred to by reference number 210) run through a flow cytometer 100,250 to determine the levels of compensation, such as shown in FIG. 2B. Single staining of the particles 210A-210E can reveal the respective spectral profile or signature 212A-212E of respective fluorochromes to the fluorescent photo-detectors of the instrument. The information obtained from the single stained particles 210 can be subsequently used to determine a simplicity index and a complexity index of a set of fluorochromes attached to the particles 210. The information obtained from the single stained particles 210 can also be subsequently used to determine a reference full spectrum or spectral signature for a fluorochrome useful for unmixing data from a mixed sample labeled with multiple fluorochromes.

The staining of the compensation control usually should be as bright or brighter than the sample. Antibody capture beads can be substituted for cells and one fluorophore conjugated antibody for another, if the fluorescence measured is brighter for the control. The exceptions to this are tandem dyes, which cannot be substituted. Tandem dyes from different vendors or different batches must be treated like separate dyes, and a separate single-stained control should be used for each because the amount of spillover may be different for each of these dyes. Also, the compensation algorithm should be performed with a positive population and a negative population. Whether each individual compensation control contains beads, the cells used in the experiment, or even different cells, the control itself must contain particles with the same level of auto-fluorescence. The entire set of compensation controls may include individual samples of either beads or cells, but the individual samples must have the same carrier particles for the fluorophores. Also, the compensation control uses the same fluorophore as the sample. For example, both green fluorescent protein (GFP) and Fluorescein isothiocyanate (FITC) emit mostly green photons, but have vastly different emission spectra. Accordingly, the system cannot use one of them for the sample and the other for the compensation control. Also, the system must collect enough events to make a statistically significant determination of spillover (e.g., about 5,000 events for both the positive and negative population).

During calibration in a conventional flow cytometer, the system obtains an initial spillover matrix from single stained reference controls. In a conventional flow cytometer, the fluorescence signals (e.g., colors) are separated out into discrete fluorescent bands using a series of edge filters and dichroic mirrors. The system detects (e.g., measures) each individual channel with a photo multiplying tube (PMT). During detection of the fluorescent signals, “spillover” can occur between fluorescent bands, which ideally are completely discrete, such as shown in the combined profile 226. The system defines the spillover (e.g., spillover 228 in the combined profile 226 in FIG. 2C) between the fluorescent bands with a spillover matrix [S].

Alternatively, during calibration in a spectral flow cytometer, the system obtains an initial reference matrix from single stained reference controls 210. Spectral flow cytometry is a technique based on conventional flow cytometry where a spectrograph and multichannel detector (e.g., charge-coupled device (CCD)) is substituted for the traditional mirrors, optical filters and photomultiplier tubes (PMT) in conventional systems. In the spectral flow cytometer, the side scattered light and fluorescence light is collected and coupled into a spectrograph, either directly or through an optical fiber, where the whole light signal is dispersed and displayed as a high-resolution spectrum on the CCD or coupled into one or more multichannel detectors for detection.

In process step 204 of FIG. 2A, the sample 220 shown in FIG. 2C is run through the flow cytometer 100,250. The sample 220 includes a plurality of marked cells or particles 222A-222E that flow through each laser beam of each laser and generates fluorescent light and/or scattered light referred to as an event. The fluorescent light and/or scattered light is captured and detected in order to identify the particle and generate counts for the various types of particles in the sample 220. For each particle in the sample fluid 210 passing by the laser beam(s) and fluorescing light and/or scattering light, the system generates, obtains, and/or records data (e.g., event data) representing the overall spectral profile 226. For example, fluoresced cells in the sample fluid flowing through the flow cytometer are detected. An event occurs per particle/cell. Each full spectrum or spectral detection of a fluoresced cell by the detector modules excited by the lasers is an event. The event data for a particle/cell may be defined according to a measured sample event vector.

In step 205, the system generates a compensated sample event vector (for conventional flow cytometer) or an unmixed sample event vector (for spectral flow cytometer) to count the number of various types of cells or particles in a sample 222 to obtain a measure of concentration. Generally as shown in FIG. 2D, an inverse matrix 234 (determined from the initial spillover matrix and/or the initial reference matrix with fine adjustments) is used on the event data representing the spectral profile 226 to generate the compensated sample event vector or the unmixed sample event vector representing separate spectral profiles or signatures 236A-236E of the various auto-luminescence (generated by the cells or particles themselves) or luminescence given off by the fluorochromes tagged to the various cells 222A-222E in the sample 220. For the conventional flow cytometer, the system calculates the compensated event vector based on the initial spillover matrix and the measured sample event vector. For the spectral flow cytometer, the system calculates the unmixed sample event vector based on the initial reference matrix and the measured sample event vector.

Unfortunately, the initial spillover matrix and the reference matrix tend to be insufficiently accurate to yield reliable results. An additional step can be taken, a fast compensation step, which includes compensating for inaccuracies of the initial spillover matrix and/or the reference matrix. Subsequently thereafter, based on the fast compensation, the system generates can generate a re-compensated sample event vector.

Obtaining Spillover Matrix from Single Stain Controls

A conventional flow cytometer generates or obtain a spillover matrix from single stained controls. A spectral flow cytometer can similarly obtain a spillover matrix. The steps for generating or obtaining a spillover matrix by using a conventional flow cytometer are further discussed.

Assume matrix [S] is an N×N dimensional spillover matrix obtained from single stained compensation controls, where N is the number of fluorescent detectors. Example compensation controls include beads 210 stained or dyed with fluorochromes such as fluorescein isothiocyanate (FITC), R-phycoerythrin (PE), Peridinin Chlorophyll Protein Complex (PerCP), phycoerythrin and cyanine dye (PE-Cy7), Allophycocyanin (APC), and a tandem fluorochrome combining APC and cyanine dye (APC-Cy7).

Assume vector {U} is a measured sample event vector with N values, each of which is from one of the N detectors detecting a compensation control (e.g., FITC, PE, PerCP, PE-Cy7, APC, APC-Cy7).

Assume vector {V} is the compensated sample event vector with N values. The measured sample event vector {U} is equal to the spillover matrix [S] multiplied with the compensated sample event vector {V}. This can be represented with the following matrix relationship with the measured sample event vector {U}:

$$[ S ] \{ V \} = \{ U \} \qquad Eq . 1$$

Therefore, with the inverse spillover matrix [S]⁻¹, the compensated sample event vector {V} can be obtained from the matrix equation:

$$i ) = \{ V \} = {[ S ]}^{- 1} \{ U \} \qquad Eq . 2$$

An initial spillover matrix [S] can be obtained by measuring each single stained control (e.g., FITC, PE, PerCP, PE-Cy7, APC, APC-Cy7) at each detector to obtain the following matrix:

$$1. [ S ] = [ \begin{matrix}1. & {S}_{1 , 2} & … & {S}_{1 , n} \\ {S}_{2 , 1} & 1. & … & {S}_{2 , n} \\ ⋮ & ⋮ & ⋮ & ⋮ \\ {S}_{n , 1} & {S}_{n , 2} & … & 1.\end{matrix} ] \qquad Eq . 3$$

In the subscript x,y in Eq. 3, the x value represents the detector number. The y value of the subscript x,y in Eq. 3 represents the column associated with a single stained control.

Each column in the initial spillover matrix [S], a separation of variables (SOV) matrix, corresponds to one single stained control (e.g., FITC, PE, PerCP, PE-Cy7, APC, APC-Cy7). For example, column one corresponds to FITC single stained control. As another example, column two corresponds to PE single stained control; and so on for each single stained control that is run to calibrated the flow cytometer. Each row in the initial spillover matrix [S] corresponds to a given detector number. For example, row one corresponds with detector 1. Row two corresponds to detector 2, and so on.

In general, the initial spillover matrix that is generated is not accurate enough to accurately separate spectrum and identify cells or particles. Accordingly, fine adjustment of the non-diagonal element values of the initial spillover matrix [S] is needed (e.g., fine adjustment to the initial spillover matrix [S] generating an adjusted spillover matrix [S]′ and its associated inverse, the adjusted compensation matrix [C]′). The fine adjustments may be made based on experience and judgment of the lab technician/operator. The fine adjustments are often made to correct the distortion caused by either the interactions of fluorochromes stained on the same cells or particles, or by the system for the measurements of the single stained and unstained controls, or by both distortions caused by the interactions and the system. Assume an adjustment matrix [D] is the fine adjustments to be made (e.g., added) to the non-diagonal element values of the initial spillover matrix [S]. A re-compensated event vector {V_{R}} can be determined from the matrix equation {V_{R}}=[[S]+[D]]⁻¹ {U}.

### Obtaining Unmixed Event List Data for a Spectral Flow Cytometer

Alternatively, the system can include a spectral flow cytometer to generate or obtain unmixed event list data. The steps for generating or obtaining unmixed event list data by using a spectral flow cytometer are further discussed.

Assume [R] is a N×M reference matrix obtained from single stained reference controls, where N is the number of detectors, M is the number of fluorochromes ((e.g., FITC, PE, PerCP, PE-Cy7, APC, APC-Cy7) to be measured with M always less than N. In other words, the number of fluorochromes that are to be used to mark particles/cells in a mixed sample is less than the number of detectors. The matrix [R] is a set of full spectrum or spectral signatures obtain by independent runs of the single stained reference control for each fluorochrome that is to be used to label particles/cells in a mixed sample.

Assume {U} is a measured sample event vector with N values, each value of intensity is from one of the N detectors over a predetermined range of wavelengths. The measured sample event vector is obtained by running the labeled mixed sample with particles/cells that were labeled with the M fluorochromes.

Assume {V} is the unmixed sample event vector with M values (e.g., fluorescence intensity), each of which is the unmixed value for a fluorochrome (e.g., one of the FITC, PE, PerCP, PE-Cy7, APC, APC-Cy7).

The unmixed sample event vector {V} has the following matrix relationship with the measured sample event vector {U}:

$$[ R ] \{ V \} = \{ U \}$$

Since the number of the variables M in the unmixed sample event vector {V} is less than the number of variables N in the measured sample event vector {U} (e.g., the dimension of the unmixed sample event vector is less than the dimension of the measured sample even vector), then the system uses a least square algorithm to obtain the solution of the above equation.

Compared with conventional flow cytometer, the unmixed sample event vector is equivalent to the compensated event vector. Therefore, the spectral spillover matrix [S] for the unmixed event list data (e.g., unmixed sample event vector) is an identity matrix [I] as follows:

$$i ) [ S ] = [ \begin{matrix}1. & 0 & … & 0 \\ 0 & 1. & … & 0 \\ ⋮ & ⋮ & ⋮ & ⋮ \\ 0 & 0 & … & 1.\end{matrix} ]$$

In general, the unmixed event list data is not accurate enough so that fine adjustment of identity spectral spillover is needed (e.g., fine adjustment to generate an adjusted spectral spillover matrix). Accordingly, the equation for the re-compensated event vector becomes {V_{R}}=[[I]+[D]]⁻¹ {V} where [D] is an n×n delta matrix with fine adjustments δ_{i,j} in the i^{th} row and j^{th} column respectively and zeroes where no fine adjustment is needed. For example, a delta matrix can be

$$[ D ] = [ \begin{matrix}{δ}_{1 , 1} & ⋯ & 0 & ⋯ & {δ}_{1 , n} \\ {δ}_{2 , 1} & ⋯ & ⋮ & ⋯ & {δ}_{2 , n} \\ ⋮ & ⋯ & ⋮ & ⋯ & ⋮ \\ 0 & ⋯ & {δ}_{i , j} & ⋯ & 0 \\ ⋮ & ⋯ & ⋮ & ⋯ & ⋮ \\ {δ}_{n , 1} & ⋯ & 0 & ⋯ & {δ}_{n , n}\end{matrix} ] .$$

### Fast Compensation of Flow Cytometry Data

Accordingly, in flow cytometry (e.g., conventional and spectral), Flow Cytometry Standard (FCS) data collected from a cytometer is linear raw list data. The list data needs to be compensated before it is consumed on plots and used for statistics analysis. The system performs fast compensation to account for insufficient accuracies in a spillover matrix and/or unmixed event list data.

Compensation of list data is based on an initial spillover matrix that the system obtains from measured single stained compensation controls and/or from fine adjustment input. The obtained initial spillover matrix is in general not accurate enough. Fine adjustments are made that generate an adjusted spillover matrix by finely adjusting values in the initial spillover matrix.

Every time a spillover value is finely adjusted, the spillover matrix needs to be inverted to obtain the compensation matrix. Then the compensation matrix is multiplied by each list data event vector to generate the compensated list data (e.g., re-compensated event vector).

Take an experiment of N fluorescent parameters, for example. For the compensation of each event vector, it requires N² multiplications plus N×(N−1) additions to generate the compensated event vector. The computation complexity is on the order of N² (e.g., O(N²).

For an experiment with a limited number of fluorochrome parameters and limited number of events, compensation calculation may not be the bottleneck in flow cytometry data analysis. However, if an experiment contains a large number of fluorescent parameters (e.g., over 20 fluorescent parameters) with a large number of events (e.g., 2 million events), the compensation calculation can be extremely time consuming. The consequence is that each time the system changes a spillover value, displayed plots and statistics can be extremely slow to respond on a computer interface due to extensive amount of computations processed.

Advantageously, the present system performs a fast compensation algorithm that significantly reduces the amount of computations without sacrificing any accuracy for the compensated list data when the system receives or performs fine adjustment of the spillover matrix for flow cytometry data analysis. This fast compensation algorithm requires, for example, only (3N+1) multiplications/divisions plus (N+1) additions. The complexity of this fast compensation algorithm is on the order of N (e.g., O(N)). Therefore, the present system can significantly improve the responsiveness of the displayed plots and statistics.

Consider, for example, a 20-color experiment with one million events. Whenever the system receives or performs fine adjustment of a spillover value, a typical compensation algorithm requires a total of 400 million multiplications plus 399 million additions. In contrast, the fast compensation algorithm of the present system requires only a total of 60 million multiplications plus 20 million additions. The saving of the total multiplications and additions are 566% and 1895%, respectively, compared with a typical compensation algorithm.

The following is the derivation of the present fast compensation algorithm:

Assume matrix [C] is the compensation matrix. The compensation matrix [C] is the inverse of the spillover matrix [S] by the matrix equation [C]=[S]⁻¹. If the compensation matrix [C] and the spillover matrix [S] are multiplied together, one acquires the identity matrix such as in the matrix equation [C] [S]=[I]. The compensated event vector {V} can be computed by multiplying the compensation matrix [C] and the uncompensated measured event vector {U} together represented by the matrix equation {V}=[C] {U}.

Due to a fine adjustment, the system generates or calculates an adjusted spillover matrix [S]′. Assume the value of one element in the initial spillover matrix [S] is changed, for example S_{i,j}′=>S_{i,j}+δ_{i,j}, the finely adjusted spillover matrix [S]′ can be represented by the sum of the initial spillover matrix [S] summed with the fine adjustments in the delta matrix [D] by the matrix equation [S]′=[S]+[D] where

$$[ D ] = [ \begin{matrix}0 & ⋯ & 0 & ⋯ & 0 \\ ⋮ & ⋯ & ⋮ & ⋯ & ⋮ \\ ⋮ & ⋯ & ⋮ & ⋯ & ⋮ \\ 0 & ⋯ & {δ}_{i , j} & ⋯ & 0 \\ ⋮ & ⋯ & ⋮ & ⋯ & ⋮ \\ 0 & ⋯ & 0 & ⋯ & 0\end{matrix} ]$$

is the delta matrix in which subscripts i and j represent the i^{th} row and j^{th} column respectively. The re-compensated event vector {V_{R}} can be calculated by multiplying the inverse of the finely adjusted spillover matrix [S]′, the finely adjusted compensation matrix [C]′, and the uncompensated measured event vector {U} together such as represented by the matrix equation {V_{R}}=[S]+[D]⁻¹ {U}. The delta matrix [D] has the same dimensions as the initial spillover matrix [S]. The delta matrix [D] includes delta values δ_{i,j} for finely adjusting the initial spillover matrix [S].

Since [S]+[D]=[S] ([I]+[C] [D]), [S]+[D]⁻¹=([I]+[C] [D])⁻¹[C], the equation for the re-compensated event vector {V_{R}} can be rewritten as

$$( a ) \{ {V}_{R} \} = {( [ I ] + [ C ] [ D ] )}^{- 1} [ C ] \{ U \} = {( [ I ] + [ C ] [ D ] )}^{- 1} \{ V \}$$

where ([I]+[C] [D])⁻¹ is a re-compensation matrix.

Since the re-compensation matrix can be simplified as

$${( [ I ] + [ C ] [ D ] )}^{- 1} = {[ \begin{matrix}1 & ⋯ & {C}_{1 , i} {δ}_{i , j} & ⋯ & 0 \\ ⋮ & ⋯ & ⋮ & ⋯ & ⋮ \\ ⋮ & ⋯ & ⋮ & ⋯ & ⋮ \\ 0 & ⋯ & 1 + {C}_{j , i} {δ}_{i , j} & ⋯ & 0 \\ ⋮ & ⋯ & ⋮ & ⋯ & ⋮ \\ 0 & ⋯ & {C}_{n , i} {δ}_{i , j} & ⋯ & 1\end{matrix} ]}^{- 1} = [ \begin{matrix}1 & ⋯ & \frac{- {C}_{1 , i} {δ}_{i , j}}{1 + {C}_{j , i} {δ}_{i , j}} & ⋯ & 0 \\ ⋮ & ⋯ & ⋮ & ⋯ & ⋮ \\ ⋮ & ⋯ & ⋮ & ⋯ & ⋮ \\ 0 & ⋯ & \frac{1}{1 + {C}_{j , i} {δ}_{i , j}} & ⋯ & 0 \\ ⋮ & ⋯ & ⋮ & ⋯ & ⋮ \\ 0 & ⋯ & \frac{- {C}_{n , i} {δ}_{i , j}}{1 + {C}_{j , i} {δ}_{i , j}} & ⋯ & 1\end{matrix} ] ,$$

then the matrix equation for the re-compensated event vector can be written as

$$\{ {V}_{R} \} = [ \begin{matrix}1 & ⋯ & \frac{- {C}_{1 , i} {δ}_{i , j}}{1 + {C}_{j , i} {δ}_{i , j}} & ⋯ & 0 \\ ⋮ & ⋯ & ⋮ & ⋯ & ⋮ \\ ⋮ & ⋯ & ⋮ & ⋯ & ⋮ \\ 0 & ⋯ & \frac{1}{1 + {C}_{j , i} {δ}_{i , j}} & ⋯ & ⋮ \\ ⋮ & ⋯ & ⋮ & ⋯ & ⋮ \\ 0 & ⋯ & \frac{- {C}_{n , i} {δ}_{i , j}}{1 + {C}_{j , i} {δ}_{i , j}} & ⋯ & 1\end{matrix} ] = [ \begin{matrix}{V}_{1} \\ ⋮ \\ {V}_{j} \\ ⋮ \\ ⋮ \\ {V}_{n}\end{matrix} ] = [ \begin{matrix}{V}_{1} - {V}_{j} \frac{{C}_{1 , i} {δ}_{i , j}}{1 + {C}_{j , i} {δ}_{i , j}} \\ ⋮ \\ {V}_{j} - {V}_{j} \frac{{C}_{j , i} {δ}_{i , j}}{1 + {C}_{j , i} {δ}_{i , j}} \\ ⋮ \\ ⋮ \\ {V}_{n} - {V}_{j} \frac{{C}_{n , i} {δ}_{i , j}}{1 + {C}_{j , i} {δ}_{i , j}}\end{matrix} ]$$

Each component of the re-compensated vector is determined by an addition/subtraction and multiplication/division with components of the uncompensated measured event vector {U} thereby significantly reducing the number of computations. Accordingly, the re-compensated event vector {V_{R}} can be computed much more quickly by a processor of a computer using the fast compensation algorithm.

Thus, using the fast compensation algorithm, calibration bead samples can be more quickly analyzed with a flow cytometer and results more efficiently obtained. Instead of a researcher or a lab technician spending one or more days to obtain data, data can be obtained within hours by using the fast compensation algorithm.

### Full Spectrum Flow Cytometer

Referring now to FIGS. 2E1-2E2, a schematic diagram of a full spectrum flow cytometer 250 is shown. United States (US) patent application Ser. No. 15/659,610 titled COMPACT DETECTION MODULE FOR FLOW CYTOMETERS filed on Jul. 25, 2017 by inventors Ming Yan et al., and U.S. patent application Ser. No. 15/498,397 titled COMPACT MULTI-COLOR FLOW CYTOMETER filed on Apr. 26, 2017 by David Vrane et al. describes further details of flow cytometers and are incorporated herein by reference.

The full spectrum flow cytometer 250 can be variably configured with different numbers of lasers and different numbers of detector modules. In one embodiment, the full spectrum flow cytometer 250 can include five lasers (Red 640 nm, Yellow-Green 561 nm, Blue 488 nm, Violet 405 nm, and UV 355 nm) 251A-251E and five detector modules 252A-252E as shown in FIG. 2E to provide full spectrum analysis. With five detector modules, each of the detector modules (Red, Yellow-Green, Blue, Violet, and UV) 252A-252E can be associated with one of the five lasers as shown in FIG. 2E. Each of the five lasers generate laser light of five different wavelengths such as ultraviolet (UV) 355 nm, Violet 405 nm, Blue 488 nm, Yellow Green 561 nm, and Red 640 nm. Equipped with five lasers and five detectors, the full spectrum flow cytometer 250 can be used to develop color panels with 28 or more colors.

The optical paths of the laser light for each of the five lasers (UV 355 nm, Violet 405 nm, Blue 488 nm, Yellow Green 561 nm, and Red 640 nm) is shown in FIG. 2E. The lasers are spatially separated, each having an independent optical path to the flow cell 255. One or more optical components 254, such as mirrors, lenses, and filters, can be used to direct the laser light of each laser into the flow cell 255 to strike particles/cells in the sample fluid as they pass by an interrogation region.

After striking a particle in the flow cell 255, the fluorescent light is collected and directed through a plurality of optical fibers 257 and one or more optical elements (e.g., lenses) 258 into each of the individual detector modules 252A-252E. Each of the detector modules 252A-252E uses a sequential array of a plurality of avalanche photodiodes (APD) as the photodetectors. The full spectrum flow cytometer 250 can further include a plurality of scatter detectors, including a forward scatter (FSC) detector 256A near the flow cell, a blue side scatter detector 256B near the lens/filters for the red detector module, and a violet side scatter detector 256C near the lens/filters for the blue detector module. The plurality of scatter detectors are typically used to control data capture by the detector modules in the flow cytometer and data storage in a storage device. Each of the detector modules 252A-252E can capture a plurality of raw digital data for a given particle/cell as each laser beam of the plurality of lasers strike the same particle. The plurality of raw digital data is captured at slightly different times (laser delay) as the marked particle/cell passes by each laser beam in the flow channel. For example, the yellow/green laser may first strike the particle generating a first set of raw digital data, the violet laser second generating a second set of raw digital data, the blue laser third generating a third set of raw digital data, the red laser fourth generating a fourth set of raw digital data, and the UV laser lastly generating a fifth set of raw digital data for the same particle. If the plurality of lasers are arranged in a different order along the flow channel, the sequential order of generation of raw digital data by the same particle will be different. While an associated detector module is capturing light from its associated lasers, data from detectors in the other detector modules can be ignored. For example, at the time when the red laser strikes the particle/cell, the data from the red detector module is captured while the data from the UV, violet, yellow green, and blue detector modules can be ignored.

With the addition of the UV laser 251A and having five detector modules providing sixty-four (64) fluorescence detectors (see FIG. 2G), the full spectrum flow cytometer 250 has the power to take highly multiplexed assays beyond thirty (30) colors. The incorporation of the UV laser 251A allows the full spectrum flow cytometer 250 to perform at a different wavelength and discriminate different colors than those systems without. The UV laser enables the use of UV light excited fluorochromes, such as BUV737 and BUV395 fluorochromes, giving researchers additional flexibility on how they design experiments for a sample of particles.

FIGS. 2F-1 to 2F-2 illustrates the configuration of each photo-detector in each of the five detector modules 252A-252E used in the embodiments of a full spectrum flow cytometer 250. Each detector has a bandpass filter in front of it to filter out light. The bandpass filter allows predetermined wavelengths through to the photo detector for detection while filtering out other wavelengths. The detector number (also referred to herein as channel number) and wavelength information of the bandpass filters associated with each photo-detector is shown. The ultraviolet (UV) detector module 252E has sixteen (16) detectors labeled as channels UV1-UV16 based on their position in the sequential array of detectors in the module. The violet detector module 252D has sixteen (16) detectors labeled as channels V1-V16 based on their position in the sequential array of detectors in the module. The blue detector module 252C has fourteen (14) detectors labeled as channels B1-B14 based on their position in the sequential array of detectors in the module. The yellow green detector module 252B has ten (10) detectors labeled as detector channels YG1-YG10 based on their position in the sequential array of detectors in the module. The red detector module 252A has eight (8) detectors labeled as detector channels R1-R8 based on their position in the sequential array of detectors in the module.

The multiple lasers in the flow cytometer are slightly spaced apart and sequentially strike the same particle/cell as it flows through the flow channel. This sets up a small amount of time delay between each subsequent laser strike (laser intercept) of the same particle/cell. There is a similar amount of time delay in the respective signal detected by the detectors and the generation of digital data from each laser strike (laser intercept) for the same particle/cell. The small amount of time is referred to as laser delay time and is predetermined by running a quality control experiment (e.g., daily QC runs) before running an experiment with a biological sample or other control. The full spectrum of fluorescence light from each laser striking the particle/cell is sent to each detector module by the fiber optic cables 257. Based on the laser delay time, the data generated by the detectors from each laser strike (laser intercept) can be associated with a given laser. For example, at one point in time a blue laser strikes the particle/cell and the detectors in the blue detector module can detect fluorescence and generate data for the blue laser strike. After a predetermined laser delay time between blue and red lasers, the same particle is struck by the red laser. Based on the time of the red laser strike, the detectors in the red detector module can detect fluorescence and generate data associated with the red laser strike. The laser delay time between the different lasers can be different but predetermined in order to be able to associate the captured data with the appropriate laser. Furthermore, the arrangement of the lasers can be in a different sequential order such that the sequence of laser strikes can differ. Moreover, the associated laser delay time can differ between laser strikes between power cycles of the flow cytometer. In any case, the data generated by each respective module that is delayed from the first data generated, is aligned together in time and associated with the particle/cell of a single event. The captured data from each detector module may be tagged with a particle/cell number count in the sample run and temporarily stored in a storage device, such as a register, memory or hard drive, for subsequent alignment together as a single event.

Fluorochromes are excited over a wavelength range (excitation wavelength range) associated with the wavelength of the laser and when excited, can emit fluorescence over a different wavelength range (emission wavelength range). The wavelength range of each detector module is associated with the expected emission wavelength range from the excitation of fluorochromes for the associated laser.

With reference to FIGS. 2F-1 and 2F-2, the bandpass filter before each detector is used to selectively pass the desirable wavelengths in the pass band range to be detected at a given photo detector for the associated excitation laser. The band bass filter rejects the wavelengths of light outside the pass band range of wavelengths. For example, the first red detector channel (R1 detector channel), the band pass filter has a center wavelength of 661 nanometers (nm) and a bandwidth of 17 nanometers around the center wavelength. Accordingly, in the band pass of wavelengths, a detector can reliably detect a wavelength range around a center wavelength and plus and minus one half the bandwidth. In the case of the R1 detector channel shown in FIG. 2F-2, the wavelength range is from the center wavelength minus one half the bandwidth (661 nm−8.5 nm=652.5 nm) to the center wavelength plus one half the bandwidth (661 nm+8.5 nm=669.5 nm). In the case of the R8 detector channel, the wavelength range is from the center wavelength minus one half the bandwidth (811.5 nm−17 nm=794.5 nm) to the center wavelength plus one half the bandwidth (811.5 nm+17 nm=828.5 nm). Accordingly, the red detector module detects fluorescent light over a wavelength range from 625 nm to 828.5 nm for fluorescent particles excited by the red laser. The yellow green detector module detects fluorescent light over a wavelength range from 567 nm to 828.5 nm for fluorescent particles excited by the yellow green laser. The blue detector module detects fluorescent light over a wavelength range from 498 nm to 828.5 nm for fluorescent particles excited by the blue laser. The violet detector module detects fluorescent light over a wavelength range from 420 nm to 828.5 nm for fluorescent particles excited by the violet laser. The ultra violet detector module detects fluorescent light over a wavelength range from 365 nm to 828.5 nm for fluorescent particles excited by the ultra violet laser. This detection range includes the full visible light (electromagnetic) spectrum from 380 nm to 780 nm, a portion (365 nm to 379 nm) of the non-visible UV light spectrum, and a portion (781 nm to 828.5 nm) of the non-visible infrared light spectrum.

If even more than 64 detectors are used, an increased granularity in the data at various wavelengths can be captured. The compactness of photo detectors (e.g., avalanche photo-diodes) and the detector array in the detector module has led to embodiments of up to 64 detectors and can lead to a further increase in the numbers of available detectors. A larger number of detectors can lead to increased numbers of colors that can be detected (discriminated) and an increased number of fluorochromes that can be used to examine particles within a single sample by a single run through a flow cytometer. The use of compact photodetectors in a compact photo detector array as the detector modules in the full spectrum flow cytometer 250 has improved the efficiency of running samples through a flow cytometer and examining the resultant data.

While a single particle has been described passing through each laser, a sample fluid run through a flow cytometer can have thousands of cells/particles per micro liter with hundreds of thousands or more of particles in a sample fluid size of hundreds of microliters (e.g. 500,000 particles in a 500 microliter sample size). The same sample can have different types of cells with hundreds of thousands or more. With a multi-color experiment, different fluorochromes are attached to different particles/cells to count different types of particles in the same sample. In a single run through the flow cytometer, the intensity and wavelength of each color of fluorescent light generated by the excited fluorochrome on the labeled cells can be detected and plotted on a chart by wavelengths to indicate the spectrum of light captured by the sample run. Furthermore, the intensity of fluorescent light for each given color/detector channel can be binned into count ranges with the particle count falling into these ranges being summed up together and plotted on the chart to show the particle cell density for the wavelengths of light.

In FIG. 2E-2, the charts 260A-260E of data, normalized intensity (Y axis) versus wavelength (X axis), represents the range of light spectral components captured by each respective detector module for all events (each cell passing through the lasers) in a sample, such as a reference control with a single fluorochrome being used to generate a reference full spectrum signature. In FIG. 2G-1, the raw channel data captured for each detector module 252A-252E can respectively be plotted, based on the detector channel number, as a portion (individual detector module spectrum signature) 261A-261E of a full spectrum (spectral) signature of the sample run. In the plots of the individual detector module spectrum signature portions 261A-261E associated with each color laser 251A-251E and associated detector module 252A-252E pairing, the intensity (Y axis) and binned density count are plotted as a function of the detector channel number (X axis). Each of the individual detector module spectrum (spectral) signatures is formed out of a channel spectrum signature, such as channel spectrum signature 265 for the detector module spectrum (spectral) signature 261D for example.

The channel spectrum signature is plotted based on a plurality of binned intensity levels and the particle counts within those bins. For example, the greatest count (highest density) at the binned intensity level range for the channel is given a first color (e.g., red) located at the center intensity level range 266 of the channel spectrum signature 265. For each channel spectrum signature, the other binned intensity levels are either above 267P, 268P, 269P or below 267M, 268M, 269M the center intensity level 266 having the greatest particle/cell count. The second intensity levels 267P,267M respectively just above 267P and below 267M the center intensity level 266 are assigned a second color differing from the first color of the center intensity level. The third intensity level 268P above the second and center intensity levels and the third intensity level 268M below the second and center intensity levels are assigned a third color differing from the first and second colors. The fourth intensity level 269P above the third, second, and center intensity levels and the fourth intensity level 269M below the third, second and center intensity levels are assigned a fourth color differing from the first, second, and third colors. In this manner, intensity density information can be communicated to the user for a given detector channel.

After generating plots of the individual detector module spectrum (spectral) signatures 261A-261E, the plots of the individual detector module spectrum (spectral) signatures can then be merged together. In FIG. 2G-2, the individual detector module spectrum (spectral) signatures 261A-261E are merged together along an X axis of detector channel number to form a plot of a full spectrum (spectral) signature 262 of the exemplary sample run through the full spectrum flow cytometer. Along the X axis, from right to left, are the red detector module spectrum signature 261A, the yellow green detector module spectrum signature 261B, the blue-detector module spectrum signature 261C, the violet detector module spectrum signature 261D, and the ultraviolet detector module spectrum signature 261E merged together forming the full spectrum signature for a given sample run. Different labeled samples run through the flow cytometer 250, will generate different detector module signatures and accordingly different merged full spectrum (spectral) signatures. Single stained control samples (reference controls) are run through the full spectrum flow cytometer used to determine the full spectrum signature of each fluorochrome before being used with other fluorochromes to label a particle/cell in a mixed sample of a plurality of particles/cells.

Instead of just looking at peak intensity levels, the full spectrum signature for one fluorochrome can be used to distinguish from noise and another fluorochrome having a different full spectrum signature. Detecting light intensity over the full spectrum is an advantage of a full spectrum flow cytometer over that of a conventional flow cytometer that just looks at peak intensity levels. When a conventional flow cytometer shows overlap in the spectrum plots of fluorescent dies, the full spectrum signatures of each when run through a full spectrum flow cytometer can be distinguishable. In planning an experiment, it is desirable to select different fluorochromes that can be distinguishable from each other by their full spectrum signatures. Fluorochromes with similar emission but different spectral signatures can be distinguished from each other. The mathematical method to differentiate between multiple fluorophores (mixed fluorescent light) is called spectral unmixing and results in an unmixing matrix that is applied to the captured data of the sample.

Particles/cells may autofluoresce when struck by the five lasers and have its own full spectrum signature. Accordingly, the autofluorescence of the various particles/cells can also be unmixed, based on the autofluorescence full spectrum signature, and be used to distinguish it from other particle/cell types and the fluorochrome attached to other cells in a mixed sample.

### Optimized Multicolor Immunofluorescence Panel (OMIP)

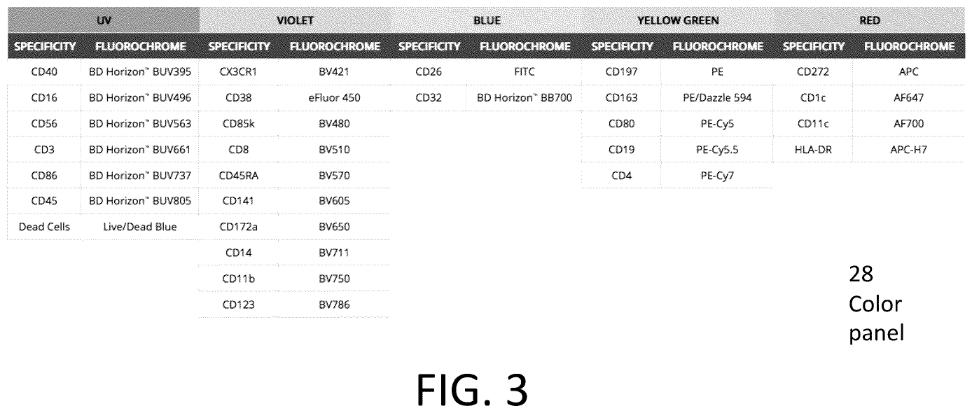

An optimized multicolor immunofluorescence panel (OMIP) generally refers to a thoroughly tested (often peer reviewed) and validated set of antibodies and reagents that can be used together for the multicolor characterization or evaluation of a specific cell state or response. An exemplary 28 color Optimized Multicolor Immunofluorescence Panel (OMIP) is illustrated in FIG. 3. The exemplary 28 color OMIP was developed using a full spectrum five laser cytometer in embodiments of the invention. Markers are listed in the SPECIFICITY columns and corresponding fluorochromes are listed under the FLUOROCHROME columns. Markers and fluorochromes are further grouped under the laser color that will optimally excite the fluorochrome.

In general, in an OMIP, the dimmest fluorochromes are assigned to antigens expressed at high levels and with high level of co-expression with other cell markers in the panel to minimize spread. Tertiary cell markers, expressed at a lower level, are assigned to bright fluorochromes to maximize resolution. For fluorochromes with the same primary excitation laser or similar emission wavelengths it is advantageous to avoid highly expressed antigens being placed in cells adjacent to co-expressed antigens with lower expression.

### 24-Color Mouse Immunoprofiling Panel

A multicolor flow cytometry panel is generally a listing of cell markers, antibodies targeting the cell markers with dyes attached to the antibodies that is used to identify biological cells of interest in a biological sample. The flow cytometry panels and immunoprofiling kits are for use with spectral flow cytometers, such as those made by Cytek Biosciences, Inc. having 3 or more lasers that include violet, blue, and red lasers.

Oftentimes, cell markers can be referred to as Specificity, Antibodies can be known as clones and dyes are called fluorochromes. Cell populations in a biological sample, via cell markers of a multicolor flow cytometry panel, can be quantified and analyzed using a full spectrum or spectral flow cytometer.

Generally, in a flow cytometry panel, each marker (antigen) is correlated to a single different fluorochrome. Generally, the dimmest fluorochrome is assigned to the highest expressing marker (antigen) and tertiary markers that express at low levels are assigned to bright fluorochromes. The exception being where multiple markers may be associated with the same fluorochrome in the case of dump channels. Dump channels or exclusion gating can be used to remove unwanted cells from a sample. For example, when a rare cell is investigated, all other cells can be labelled with a fluorochrome and then that fluorochrome can be excluded or gated out. Generally, it is desirable to avoid placing highly expressed markers adjacent to co-expressed markers with lower expression for fluorochromes with the same primary excitation laser or similar emission wavelengths.

Each marker in the panel can be detected and quantified through the collection of fluorescence emitted from its corresponding fluorochrome. A multiparametric flow cytometry panel is advantageous because a researcher can simultaneously receive multiple read-outs from a single cell, allowing for more accurate definition of cell subpopulations and profiles. This entails the need for fewer samples and/or a smaller number of cells, which is key for cells extracted from patient tissues or limited samples.

Running more markers/fluorochromes increases the complexity of the experimental design, requiring more attention in fluorochrome selection and panel design. More fluorochromes in an experiment can lead to different fluorochromes being excited by the same laser (at the same wavelength) because of overlapping emission spectra. This could lead to false positive signals since the measured signal can originate from different fluorochromes at the same time.

One of the challenges in multiparameter flow cytometry is selecting the right combination of fluorophores and antibody conjugates so that the need for compensation and spillover adjustments is kept to a minimum while the quality and accuracy of the data are not compromised. Thus, it is advantageous, to have a 24 Color Mouse Immunoprofiling Panel, that has a preselected set of fluorochromes that has already been chosen for their compatible emissions.

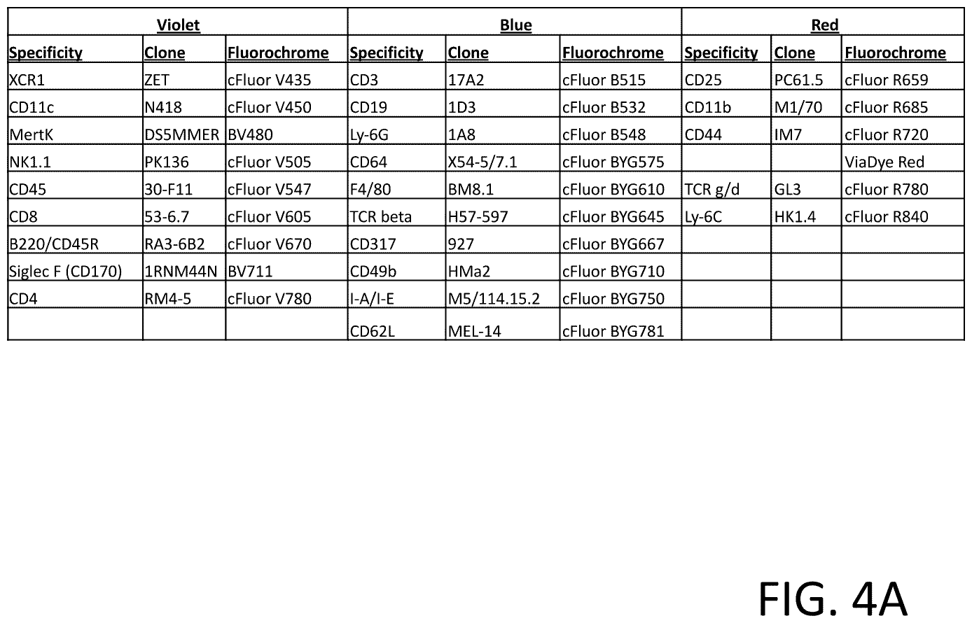

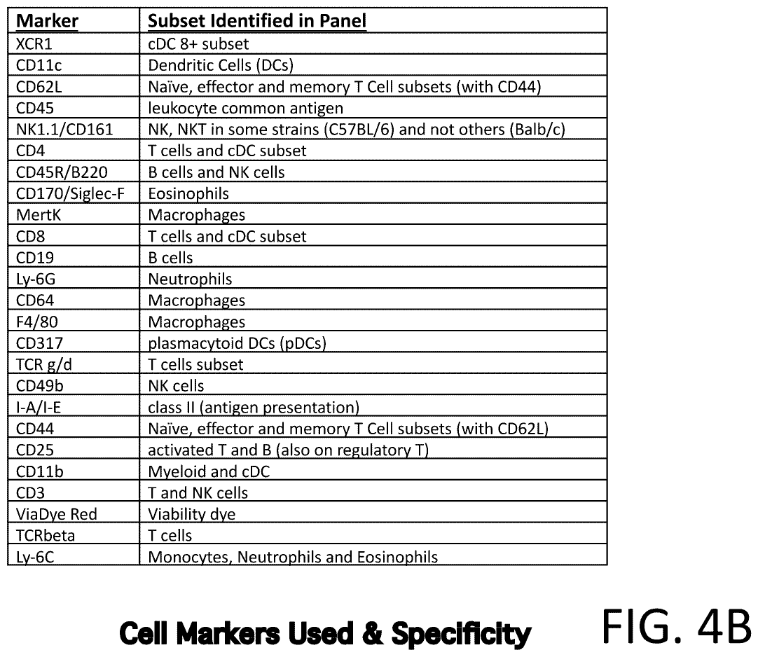

An exemplary twenty-four (24) color flow cytometry panel is detailed below. Specifically, the twenty-four (24) color flow cytometry panel uses mouse cells to provide a detailed, multi-marker analysis of immune cell populations in research models. These panels are designed to simultaneously identify and quantify a wide range of immune cells, such as T cells, B cells, NK cells, macrophages, and dendritic cells, from various mouse tissues like tumors, spleens, bone marrow, and blood. An exemplary twenty-four (24) Color Mouse Immunoprofiling Panel is illustrated in FIG. 4A with twenty-four (24) cell markers, twenty-four (24) clones, twenty-four (24) fluorochromes, and a red viability dye. From left to right the Immunoprofiling Panel comprises Violet, Blue and Red columns further subdivided into Specificity (markers), Clone (Antibodies), and Fluorochrome (Dyes) columns for each color. Specificity, clones and fluorochromes are grouped under the laser color that will optimally excite the fluorochrome. The exemplary twenty-four (24) Color Mouse Immunoprofiling Panel is illustrated in a table format of lasers (violet, blue, red), markers (specificity), clones (antibodies) and fluorochromes below. The exemplary twenty-four (24) fluorochromes are combined or attached to the twenty-four (24) antibodies or clones to form twenty-four (24) fluorochrome conjugated antibodies.

VIOLET

| SPECIFICITY/MARKER | ANTIBODY/CLONES | FLUOROCHROME |
|---|---|---|
| XCR1 | ZET | cFluor V435 |
| CD11c | N418 | cFluor V450 |
| MertK | DS5MMER | BV480 |
| NK1.1 | PK136 | cFluor V505 |
| CD45 | 30-F11 | cFluor V547 |
| CD8 | 53-6.7 | cFluor V605 |
| B220/CD45R | RA3-6B2 | cFluor V670 |
| Siglec F (CD170) | 1RNM44N | BV711 |
| CD4 | RM4-5 | cFluor V780 |

BLUE

| SPECIFICITY/MARKER | ANTIBODY/CLONES | FLUOROCHROME |
|---|---|---|
| CD3 | 17A2 | cFluor B515 |
| CD19 | 1D3 | cFluor B532 |
| Ly-6G | 1A8 | cFluor B548 |
| CD64 | X54-5/7.1 | cFluor BYG575 |
| F4/80 | BM8.1 | cFluor BYG610 |
| TCR beta | H57-597 | cFluor BYG645 |
| CD317 | 927 | cFluor BYG667 |
| CD49b | HMa2 | cFluor BYG710 |
| I-A/I-E | M5/114.15.2 | cFluor BYG750 |
| CD62L | MEL-14 | cFluor BYG781 |

RED

| SPECIFICITY/MARKER | ANTIBODY/CLONES | FLUOROCHROME |
|---|---|---|
| CD25 | PC61.5 | cFluor R659 |
| CD11b | M1/70 | cFluor R685 |
| CD44 | IM7 | cFluor R720 |
| | | ViaDye Red |
| TCR g/d | GL3 | cFluor R780 |
| Ly-6C | HK1.4 | cFluor R840 |

To enumerate the cell lineages of mouse cells, data obtained from the 24 Color Mouse Immunoprofiling Panel can be gated to separate and focus on specific cell subsets identified by their specific markers.

Now referring to FIG. 4B a table of markers and subset identified in the 24 Color Mouse Immunoprofiling Panel. The table in FIG. 4B can be referenced for quicker way to see which subset of the cell population can be quantified and analyzed by embodiments of the invention. The subset column of FIG. 34 can allow researchers to quickly determine if the 24 Color Mouse Immunoprofiling Panel is appropriate for the cell subset they wish to enumerate.

In flow cytometry, gating is the process of selecting a subset of cells from a flow cytometry experiment for further analysis. A gate is a numerical or graphical boundary that can be used to define the characteristics of particles to include for further analysis. The gating process is selecting an area on the scatter or histogram plot generated during the flow experiment that decides which cells to continue to analyze. Gating is generally done manually by sequentially selecting areas of a scatter plot generated during a flow cytometry experiment. Gating strategies are somewhat of an art form and can differ even between experts. Gating strategies can include using different types of gates such as, Forward and Side Scatter Density Plots, Forward Scatter Height vs. Forward Scatter Area Density Plot, Single Parameter Histograms, Two Parameter Density Plots, Backgating, etc.

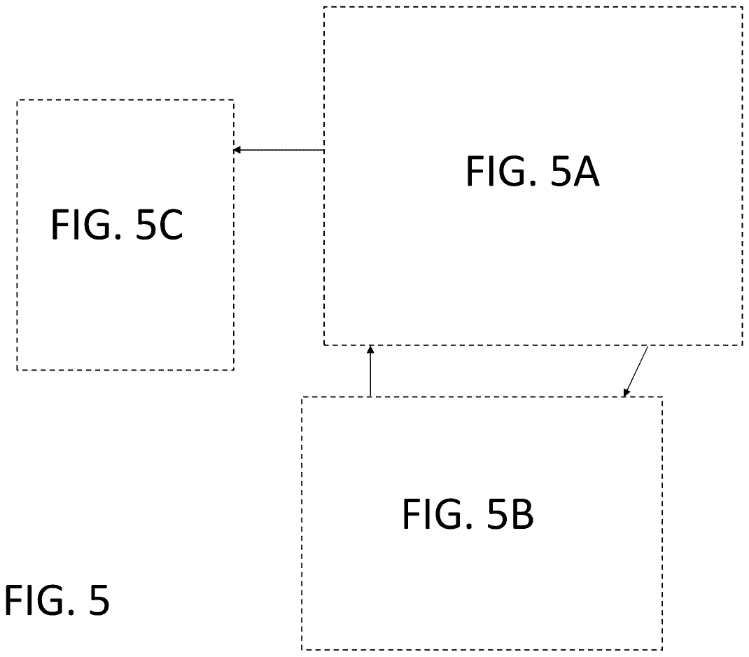

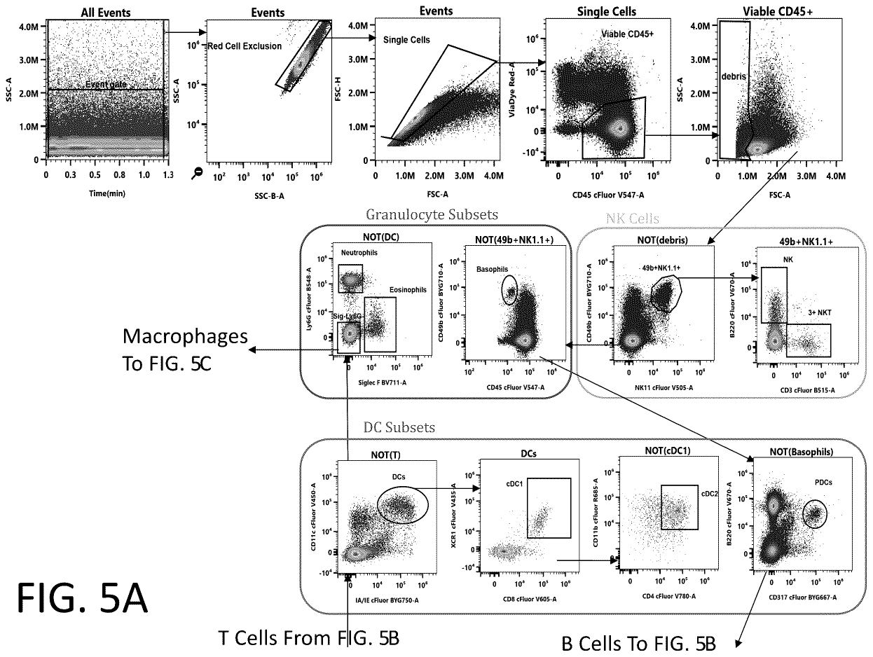

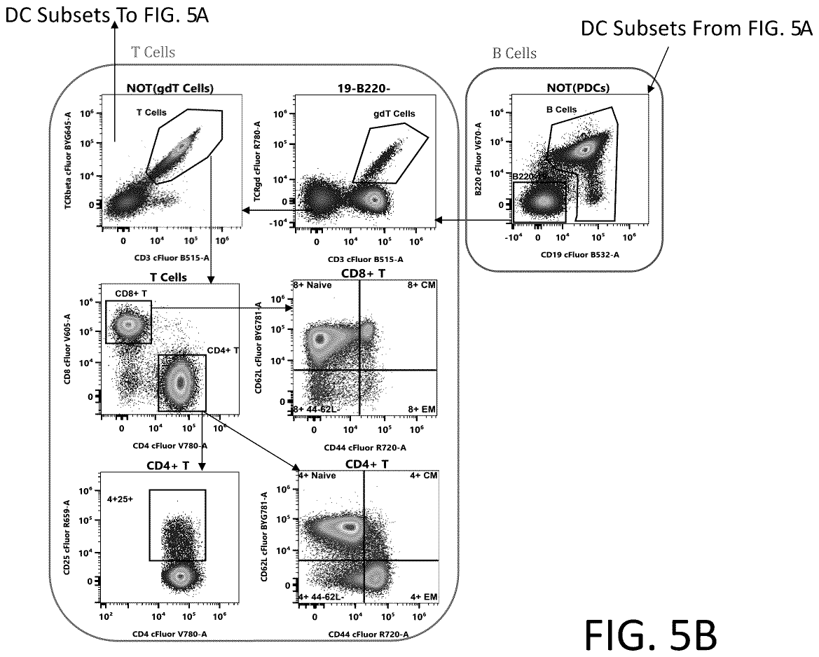

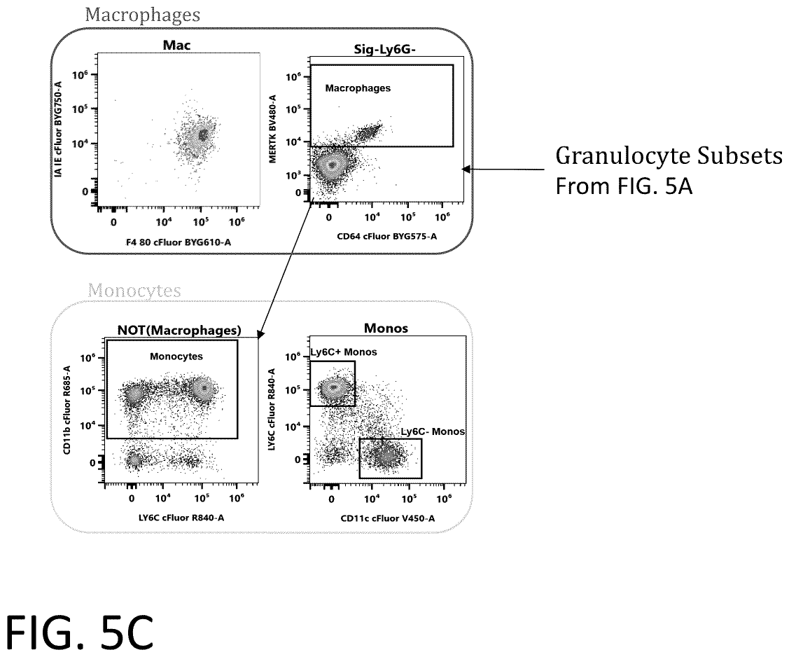

Referring now to FIG. 5 (FIGS. 5A-5C), an exemplary gating strategy for the 24 Color Mouse Immunoprofiling Panel from FIG. 4A, is described. FIGS. 5A-5C are heatmaps illustrating exemplary gating strategies for 24 Color Mouse Immunoprofiling Panel embodiment of the invention. The exemplary gating strategy illustrated in FIGS. 5A-5C are suggested for use with the 24 Color Mouse Immunoprofiling Panel and Kit. These graphs display two measurement parameters, one on the x-axis and one on the y-axis and the events are displayed as a density (or dot) plot. Each dot or point on the plot represents an individual particle that has passed through the laser. A gate has been applied to identify a specific population or to remove debris.

Data were generated using fresh splenocytes from a C57BL/6 mouse, with red blood cells lysed. Although the data was generated from splenocytes, embodiments of the invention can also be generated using other cells, such as cells found in bone marrow, lymph node and blood samples. Sequential gates of red blood cell exclusion, singlets, and viable CD45⁺ cells are used to distinguish total live splenocytes. After excluding debris from the viable CD45⁺ events, NK cells and CD3⁺ NKT-like cells are identified by expression of CD49b vs. NK1.1, and subsequently gated on CD3 vs. B220. Basophils (CD49b⁺CD45^{dim}) are identified after excluding the 49b⁺ NK1.1⁺ cells. Plasmacytoid dendritic cells (PDCs, CD317⁺⁺B220⁺) are determined after excluding the non-basophils. B cells are then identified in the non-PDC cells by positive expression of CD19 when gated vs. B220. From B220⁻CD19⁻ gated events, γδT cells are identified by co-expression of CD3 and TCRγδ. From the non-γδ-T events, T cells are gated on the CD3 vs. TCR beta plot (gating on CD3⁺TCRb⁺). CD4⁺ and CD8⁺ T cell subsets are distinguished from the total T cell gate (CD4⁺CD8⁻ and CD8⁺CD4⁻ events) and then further subdivided into naïve and memory subsets by expression of CD62L and CD44 for each T cell subset (CD4⁺ and CD8⁺). CD4⁺CD25⁺ T cells (containing activated and regulatory T cells) can be identified from the CD4 vs. CD25 plot. Dendritic cells (DCs) are gated on a CD11c vs. I-A/I-E plot from the non-T cell events. DCs are further subdivided into cDC1 and cDC2 populations using CD8 vs. XCR1 and CD4 vs. CD11b plots respectively. From non-DC cells, neutrophils and eosinophils are identified using Siglec F (CD170) vs. Ly-6G. The Siglec F (CD170)/Ly-6G double negative events are then used to classify macrophages (MERTK⁺CD64^{+/-}) from the non-macrophage events. Total monocytes are identified by expression of CD11b⁺ and are subsequently gated to determine Ly-6C⁺ and Ly-6C⁻ monocyte subsets using CD11c and Ly-6C.

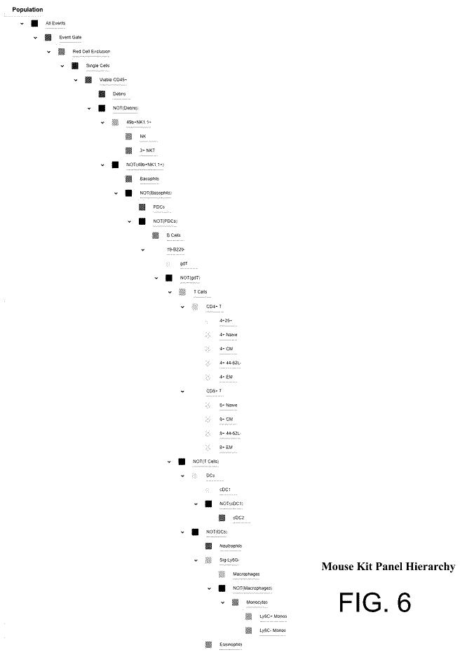

FIG. 6 illustrates the mouse kit panel hierarchy. The panel hierarchy is a drill down hierarchy display of the exemplary gating strategy described above. Panel hierarchies can be used to view the sequential steps in a gating strategy in a cleaner linear format.

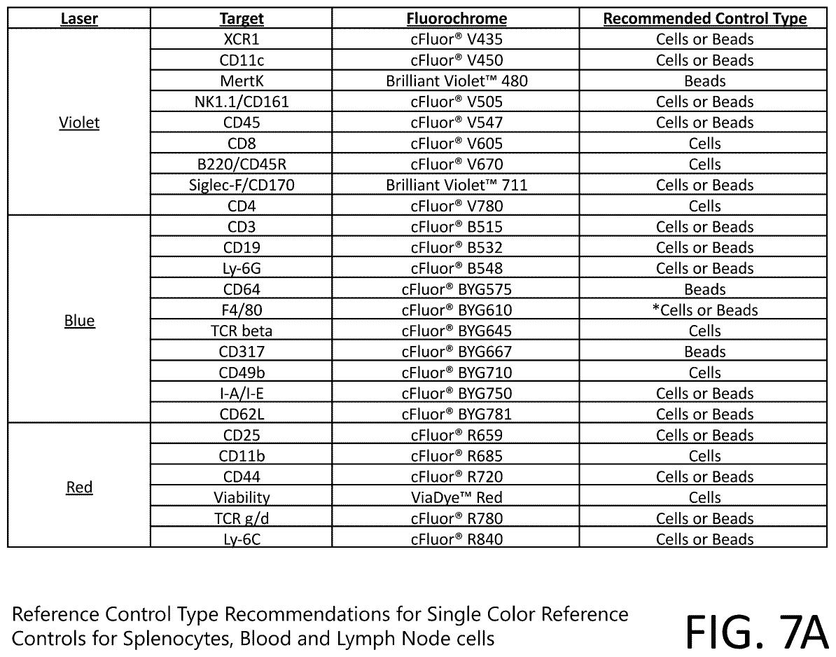

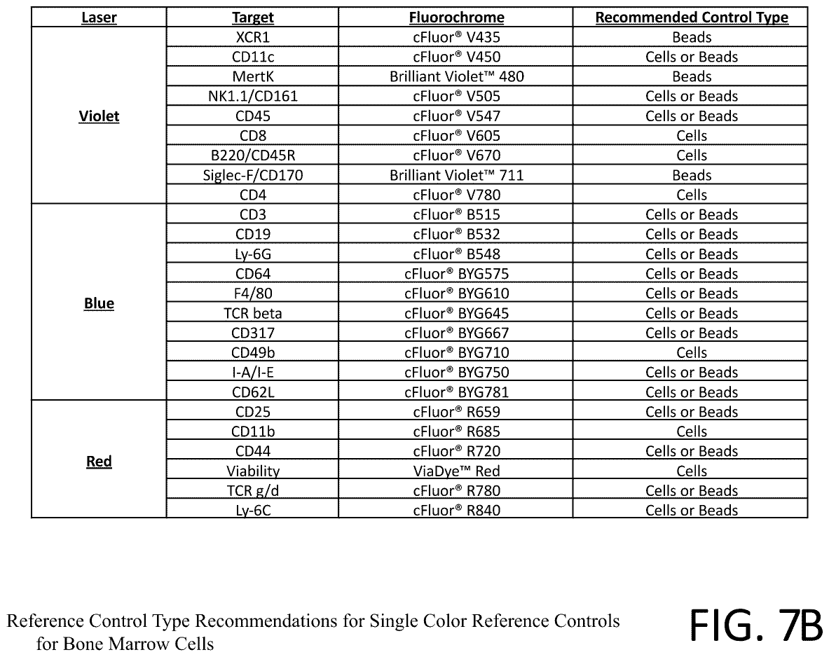

FIGS. 7A and 7B are exemplary illustrations of the 24 Color Mouse Immunoprofiling Panel with a recommended control type column. Single color reference controls can be used for unmixing. Comparing the full spectrum or spectral of the multi-color sample with the single-color reference controls enables the separation of the mixed spectra into individual fluorophores. Unmixing in flow cytometry can be the key to accurate data.

For embodiments of the invention the recommended control type, beads or cells, are listed for each marker on the “Target” column. FIGS. 7A and 7B differ in that the recommended control type can change depending on if the experimental sample is from splenocytes or bone marrow. With FIG. 7A including the recommended control type for Splenocytes and 7B including the recommended control type for bone marrow.

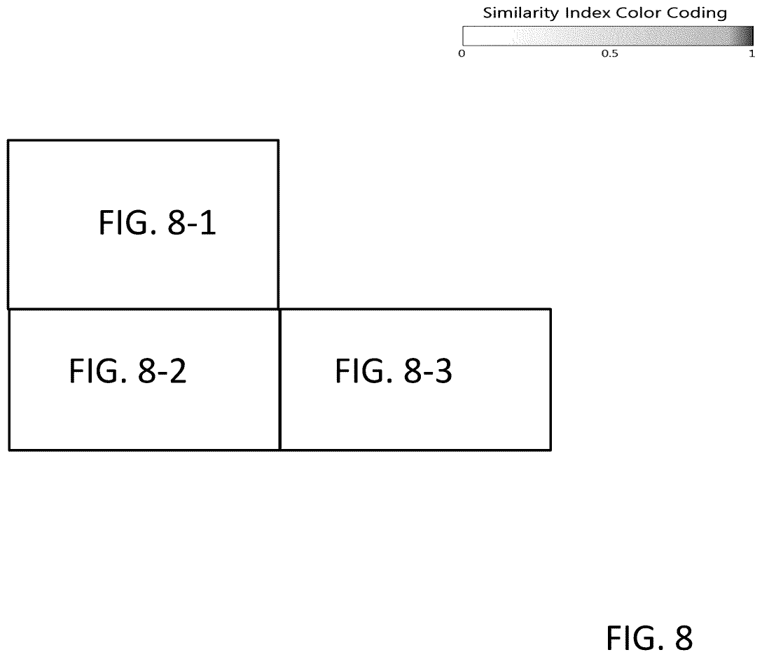

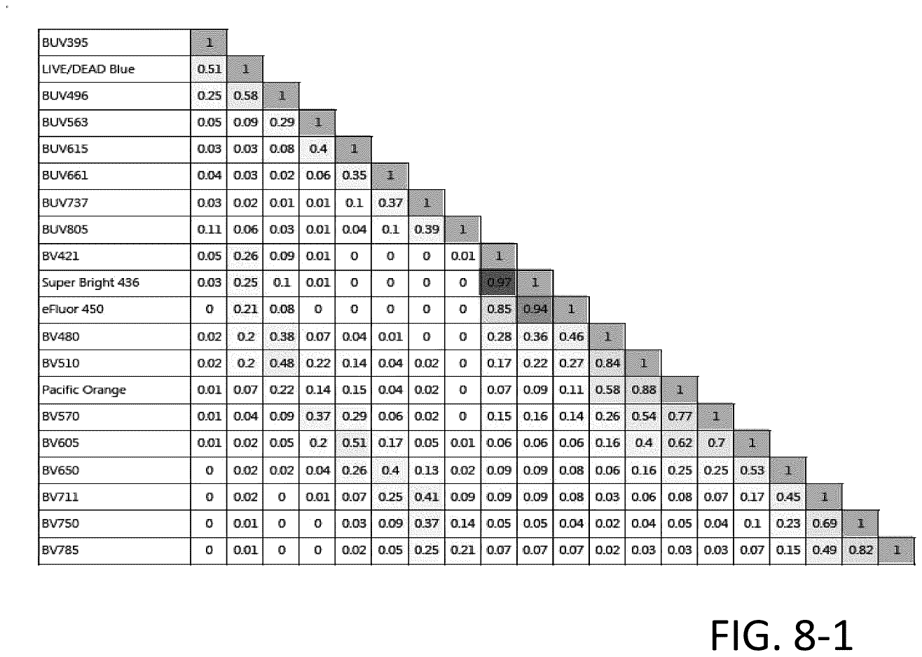

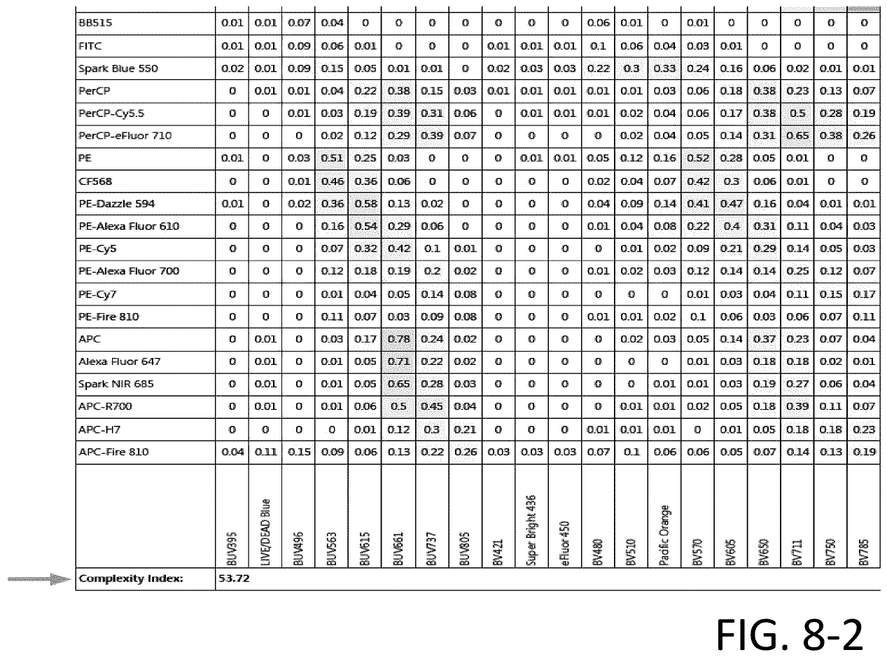

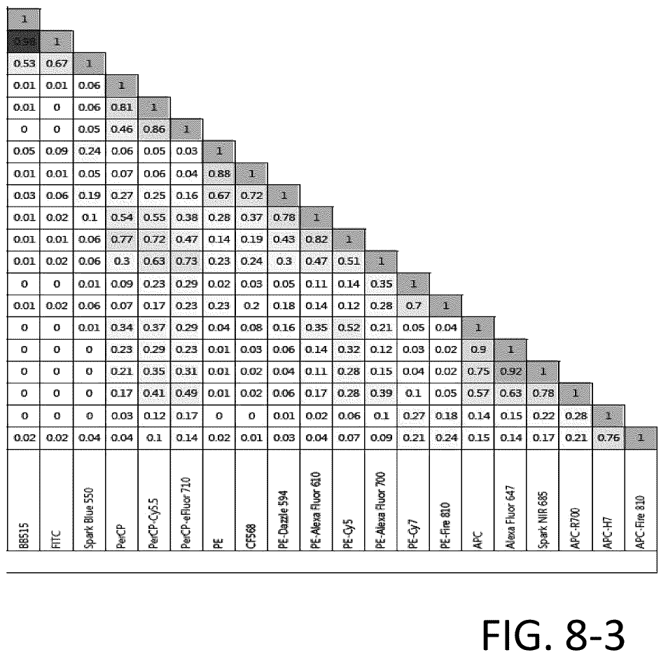

FIG. 8 (separated into FIG. 8-1 to FIG. 8-3 for purposes of reproduction) illustrates a similarity matrix for an exemplary group of forty (40) fluorochromes, sometimes simply referred to herein as colors. The similarity matrix includes a plurality of similarity indexes for pairs of each fluorochrome in the group being considered for labeling particles/cells. The similarity indexes are computed for a predetermined configuration (e.g., number of lasers, number of detector modules, number of detectors) of a flow cytometer. Because the similarity matrix is a mirror about its diagonal, only one side (upper or lower triangle of the matrix) needs to be completed. Because the diagonal is a fluorochrome paired with itself, the similarity index values for every entry along the diagonal of the matrix is the value of one (1). The value one for the similarity index indicates the fluorochrome pair along the diagonal is identical. Values of a similarity index less than one, cells off the diagonal, indicates the pairing of fluorochromes is not identical.

The cells in the similarity matrix can be color coded based on the value for similarity index being between zero and one. For example, the closer the similarity index value is to the value one, the darker color shade (e.g., dark blue) the cell in the matrix can be given. The closer the similarity index value is to the value zero, the lighter the shade of color the cell in the matrix can be given. At zero, the matrix cell is clear. The highest value of one for similarity index, can be color coded in the matrix cell with a different color (e.g., brown, red, or grey) along the diagonal. In this manner, high similarity index values and low similarity index values can be readily seen for choosing fluorochromes for a mixed sample. The respective pair of fluorochromes with high similarity index values can readily be avoided in a mixed sample or else understood in advance when used.

In FIGS. 8 (8-1 to 8-3), the value for complexity index for the set of fluorochromes is computed and displayed at the base of the similarity matrix. The complexity index is a condition number for the selected set of fluorochromes. With respect to flow cytometry, the complexity index is a measure of the multiple interferences from many fluorochromes to many fluorochromes. Stated differently, the complexity index is an overall measure of uniqueness of all dyes (fluorochromes) in a full spectrum flow cytometry panel. The lower the complexity value, the easier it will be to work with the dyes in the panel as the overall spread in the panel will be low. The higher the complexity value, the more challenging it will be to work with the selected dyes in the panel as the overall spread is high.

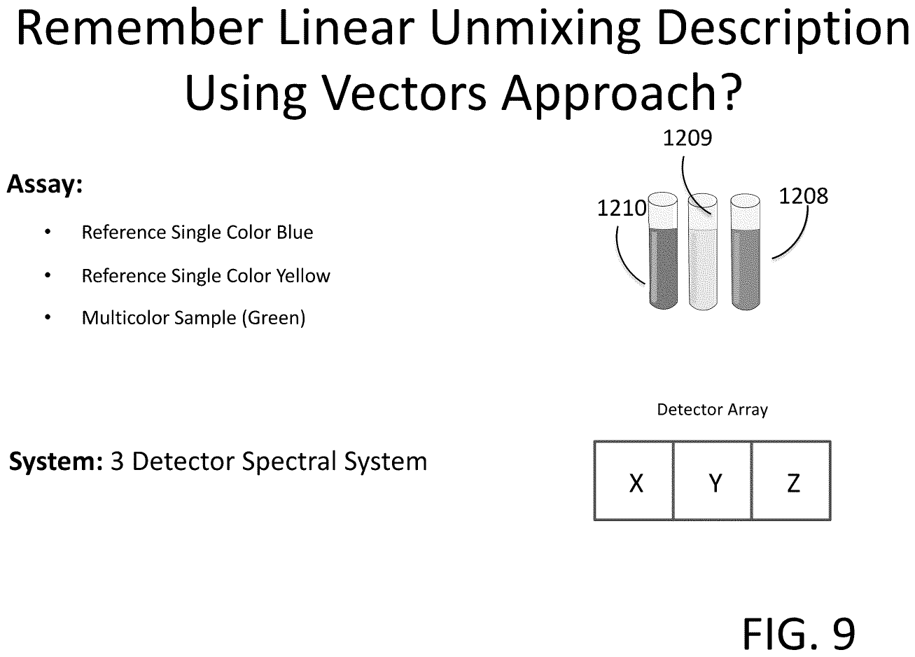

References are made to FIGS. 9-19 to illustrate how the similarity index for a pair of fluorochromes and the complexity index for a set of fluorochromes are formed and function. A number of matrices, such as the spillover matrix and others to unmix data from a mixed sample run through a full spectrum flow cytometer, are introduced in U.S. patent application Ser. No. 16/418,942; titled FAST RECOMPENSATION OF FLOW CYTOMETERY DATA FOR SPILLOVER READJUSTMENTS; filed on May 21, 2019 by Zhenyu Zhang; and incorporated herein by reference for all intents and purposes.

FIG. 9 is a simplified two-color assay (two fluorochromes) with a flow cytometer having three detectors representing only three dimensions. The two reference single colors Blue 1208 and Yellow 1209 when mixed together form a multi-sample color-green 1210. The objective is to understand how the two reference single colors interfere with each other when subsequently run together as the multi-color sample through the flow cytometer. To understand this, each reference color 1208 and 1209 is run separately through the flow cytometer and the spectral data is observed as it spills over all the detectors. Ordinarily, there is on the order of 32 detectors or multitudes thereof (e.g., 64) but such a large dimension is too difficult to simply illustrate.

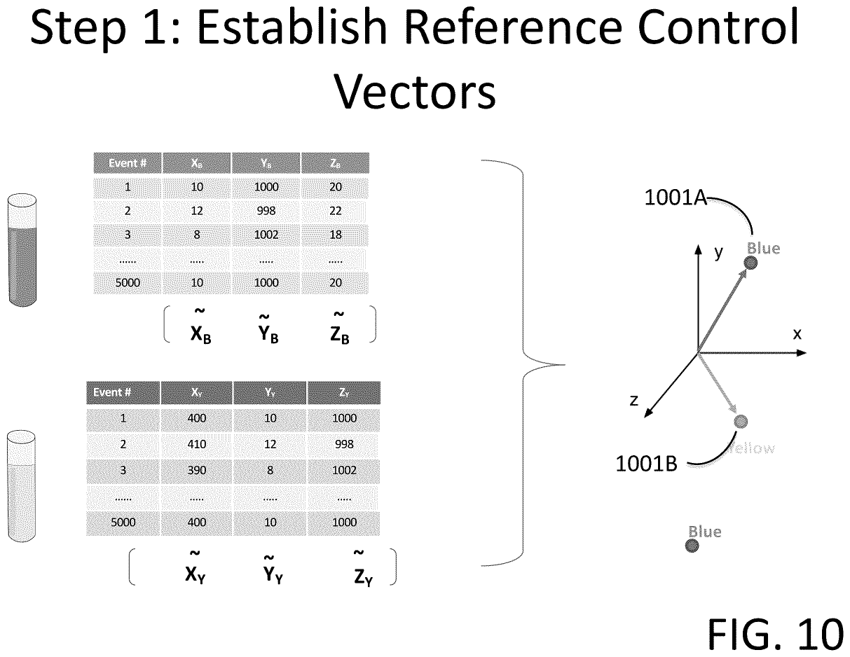

FIG. 10 illustrates the generation of reference control vectors 1001A and 1001B for each reference single colors Blue and Yellow. Five thousand events may be observed in each case representing the detection of five thousand beads or cells marked with the single fluorochrome blue in a first reference sample or a single fluorochrome yellow in a second reference sample. A blue color event vector and a yellow color event vector can be plotted in the three dimensions of the three detectors.

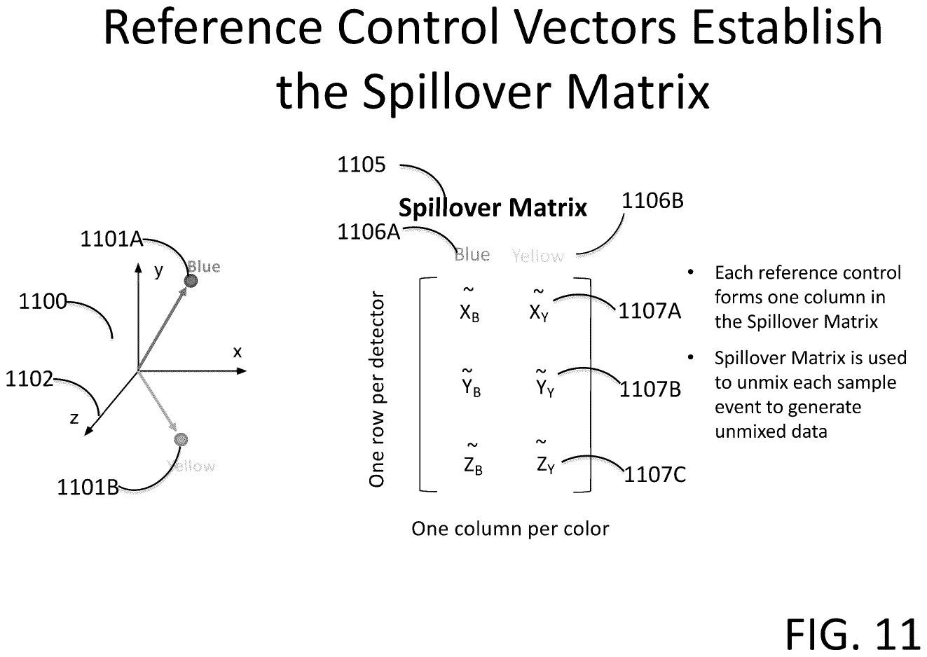

In FIG. 11, a spillover matrix 1105 can be generated from the reference control vectors 1101A and 1101B. Spillover vectors 1105 are generated and grouped together into a spillover matrix 1105. The spillover matrix 1105 is used to unmix the yellow and blue colors when the multicolor sample green is run through the flow cytometer. The spillover matrix 1105 allows events related to the yellow color and events related to the blue color to be detected from the multicolor sample when it is run through the same flow cytometer. There is a spillover vector for every column associated with each fluorochrome (color). In our example we only have two columns 1106A and 1106B, one for each single reference color in the multicolor sample. If there were 40 fluorochromes in the multicolor sample, for example, there would be 40 columns in the spillover matrix. There is a row value in each spillover vector associated with every detector in the same flow cytometer. With 64 detectors, for example, there would be 64 rows in the spillover matrix. In FIG. 11, only 3 rows 1107A-1107C are illustrated. Spillover is how one fluorochrome with a peak color in a peak channel spills over into other detectors and thereby over into other fluorochrome colors when mixed together.

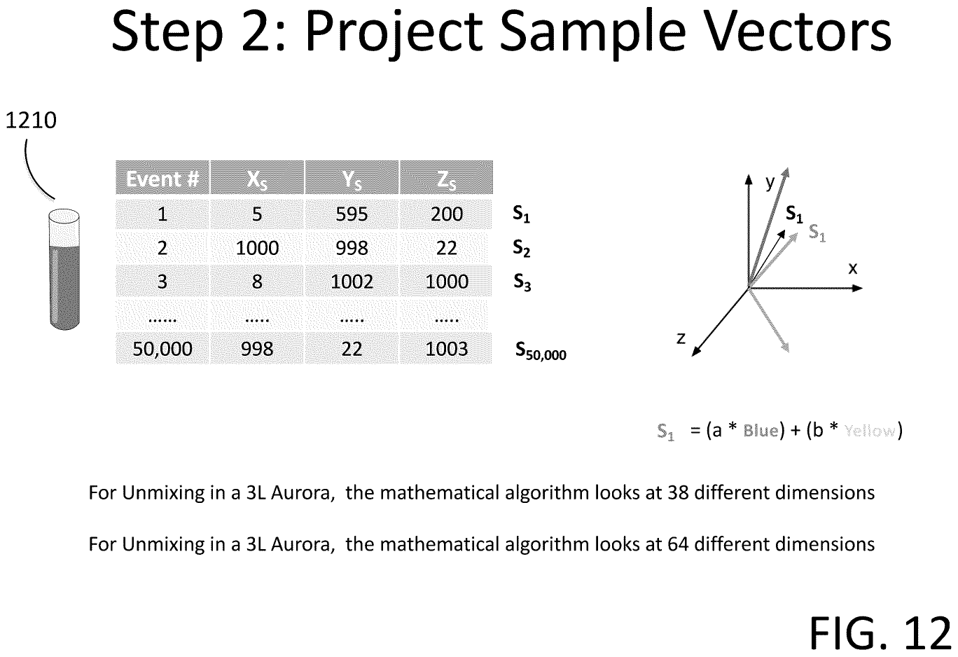

FIG. 12 illustrates a run of the multicolor sample and the generation of multicolor sample event vectors for each event representing the detection of a dye colored particle or cell. Sample 1 (green) 1210 can be unmixed by the spillover matrix to determine that it most likely represents the reference color blue. While only two fluorochromes representing two-dimensional matrix are utilized, additional dimensions can be analyzed with more lasers and more detectors. For example, up to 38 different dimensions (with 38 detectors) can be analyzed with three color excitation lasers of one flow cytometer. In another example, up to 64 different dimensions (with 64 detectors) can be analyzed with a five-color excitation laser in a different configuration of the flow cytometer.

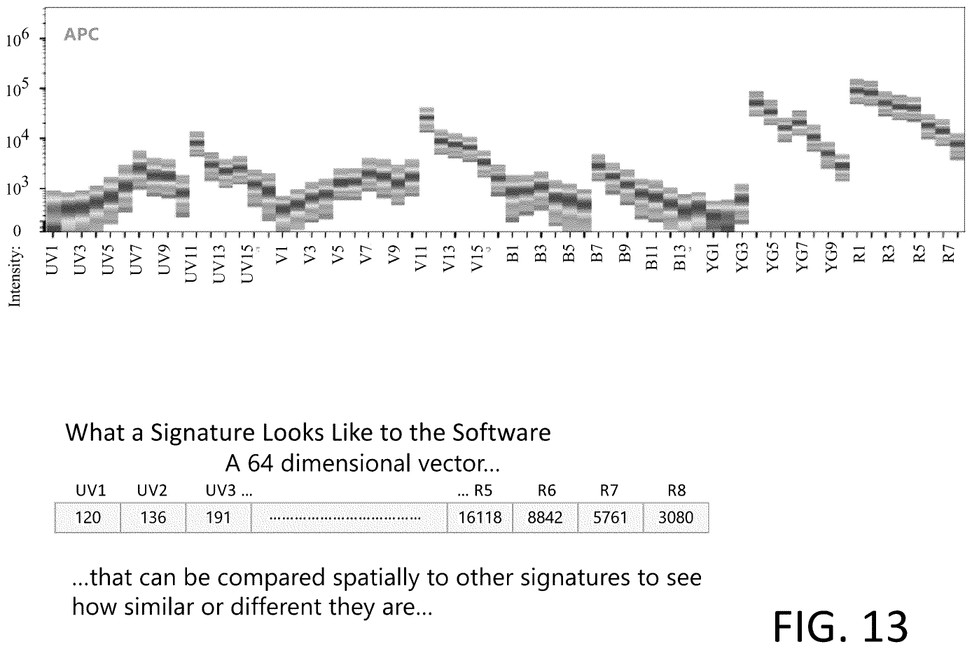

FIG. 13, top illustration, shows a full spectral signature for a 64 channel/detector flow cytometer system. FIG. 13, bottom illustration, shows a 64-dimensional vector associated with 64 detectors that mathematically represents the spectra signature shown in the top illustration. Given two spectral signature of reference samples, the spectral signature of one dye color (one fluorochrome) can be mathematically compared with the spectral signature of another dye color (one fluorochrome) to see how they overlap and interfere in advance, before they are mixed together and run through a flow cytometer. The similarity index is used to compare the reference control vectors of pairs of fluorochromes.

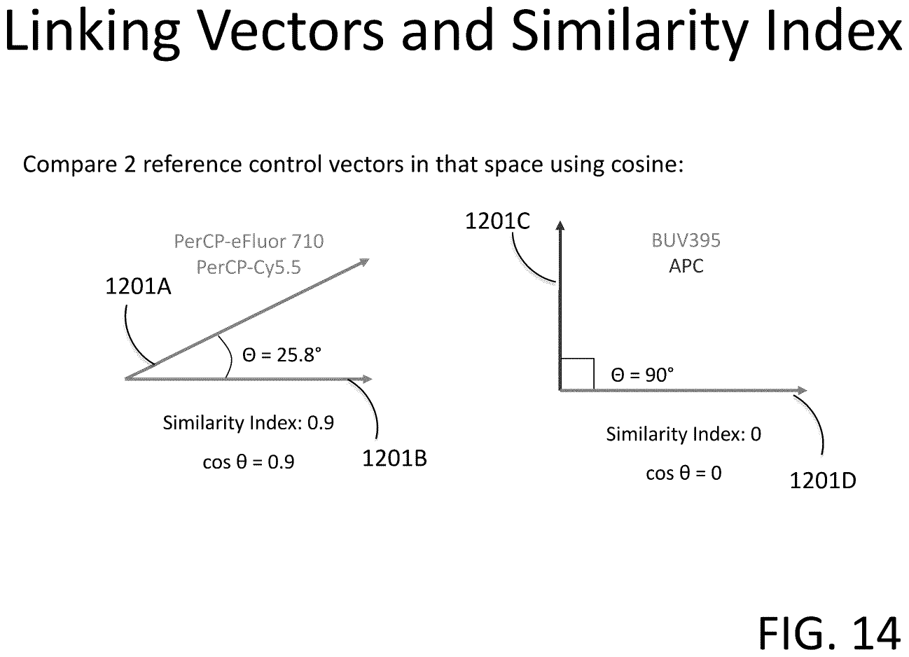

In FIG. 14, left side, an example of two reference control vectors 1201A and 1201B are plotted in two dimensions to show how a similarity index can be formed. There is a difference between the horizontal vector (vector 1-color 1) 1201B and the diagonal vector (vector 2-color 2) 1201A. That difference can be computed to show how different or how similar the refence control vectors are to each other. There are different ways to compute the difference described herein. One way is to compute the cosine of the angle theta between the two reference control vectors 1201A and 1201B. In the example illustrated on the left side, there is an angle of 25.8 degrees. Taking the cosine of this angle provides a similarity index value of 0.9. The lowest similarity index value that can produce a high resolution data has been found to be approximately 0.98. This value, or another predetermined value for each panel, that can produce high resolution data, is referred to as the threshold similarity index value. By experimentation, any larger value is not very desirable because the two dyes are too similar. Thus, it can be desirable to find and select the fluorochromes with the lowest similarity index values and reject those fluorochromes that are above the threshold similarity index value. For example, in embodiments of the invention, selecting fluorochromes can comprise first comparing the spectra of thirty-five (35) fluorochromes and assigning a similarity index to a pairing of the thirty-five (35) fluorochromes and selecting the pairs with the lowest similarity index value. In the right side of the figure, the angle between the reference control vectors 1201C and 1201D is ninety degrees. The vectors 1201C-1201D are orthogonal indicating there is no overlap. The cosine of 90 degrees is zero so the similarity index of zero represents no overlap or interference between the two selected colors. This is rather simple in two dimensions with only three detector and only two reference colors. We now have to introduce matrices to deal with the larger dimensions that are desired.

Assume a reference matrix [R] is a N by M reference matrix obtained from single stained reference controls, where N is the number of detectors, M is the number of fluorochromes to be measured with the number of fluorochromes M always being less than or equal to the number of detectors N.

Further assume that the vector {Vₘ} is a measured sample event vector with N values, with each value being from a different one of the number of detectors N of the flow cytometer.

Further assume that the vector {V_{d}} is the de-convoluted sample event vector with M values, with each value being a de-convoluted value for a different fluorochrome of the number of fluorochromes M used in a sample.

The de-convoluted sample event vector {V_{d}} can be obtained as follows:

$$\{ {V}_{d} \} = {{[ {R}^{T} R ]}^{- 1} [ R ]}^{T} \{ {V}_{m} \} or \{ {V}_{d} \} = \frac{{[ R ]}^{T}}{[ {R}^{T} R ]} \{ {V}_{m} \}$$

The de-convoluted sample event vector {V_{d}} is equal to a transpose of the reference matrix divided by the product of the transpose of the reference matrix and the reference matrix itself multiplied against the measured sample event vector {Vₘ}.

The reference matrix [R] is determined by the following equation

$$R = [ \begin{matrix}{SOV}_{1 , f 1} & {SOV}_{1 , f 2} & … & {SOV}_{1 , fM} \\ {SOV}_{2 , f 1} & {SOV}_{2 , f 2} & … & {SOV}_{2 , fM} \\ ⋮ & ⋮ & ⋮ & ⋮ \\ {SOV}_{N , f 1} & {SOV}_{N , f 2} & … & {SOV}_{N , fM}\end{matrix} ]$$

The SOV_{N,fM} values are the spillover values for each of the N detectors and each of the M fluorochromes (fM). Each fluorochrome (f1 through fM) can be run separately in a reference sample (conjugated to an antibody that is attached to a cell or a bead) through a given flow cytometer to determine the values in each column of the reference matrix [R] for each detector (1 through N) of the predetermined number of N detectors of the given flow cytometer.

### Similarity Index

In the case of the similarity index, two fluorochromes (dyes) are compared to evaluate how they interfere each other when used together in the same biological sample with markers to form a flow cytometry panel. Two reference control vectors R1 for fluorochrome 1 (f1) and R2 for fluorochrome 2 (f2) are used for example to perform a comparison.

Reference control vector

$$R 1 = [ \begin{matrix}{SOV}_{1 , f 1} \\ {SOV}_{2 , f 1} \\ ⋮ \\ {SOV}_{N , f 1}\end{matrix} ]$$

and reference control vector

$$R 2 = [ \begin{matrix}{SOV}_{1 , f 2} \\ {SOV}_{2 , f 2} \\ ⋮ \\ {SOV}_{N , f 2}\end{matrix} ] .$$

If each of the reference control vectors are plotted along lines from a center point, they would show how they diverge from each other. A difference between the two reference control vectors, such as a distance, can be used to provide a measure of interference between the two fluorochromes. There are different type of distances for above measuring purpose, such as L^{p} (Lebesgue spaces) p-norm distances of Euclidean √{(x_i−y_i)\hat{ }2)}, Minkowski √[p]{x_i−y_i)\hat{ }p)}, and Manhattan Σ{x_i−y_i}; and Cosine (from angle in between the reference control vectors). Among these distances, the Cosine of the angle between reference control vectors was more meaningful because it describes two independent controls (orthogonal reference control vectors—90-degree angle between each) when the cosine value is zero. That is, the angle between the two reference control vectors can be used as a parameter to evaluate how two dyes interfere each other in the output data of a flow cytometer when used together in the same biological sample.

Generally, the angle itself between the two reference control vectors R1 and R2 can be used to provide a measure of similarity or difference for the interference between two fluorochromes. In another case, a mathematical function (e.g., cosine function or the L^{p} p-norm distances) can be used to normalize and/or generate a measure of similarity or difference for the interference between two fluorochromes.

In accordance with one embodiment, a cosine function on the angle between the two reference control vectors is used to generate the similarity index. That is, the similarity index can be the cosine value of the angle between two spillover columns (two reference control vectors) in the reference spillover matrix R. If the similarity index is zero (cosine of 90 degrees), there is no interference between the two fluorochromes. If the similarity index is one (cosine of 0 degrees), there is complete overlap interference between the two fluorochromes because they are likely the same fluorochrome.

The similarity index is a measure of dye pair uniqueness on a scale from 0 to 1. Values close to 0 indicate that the full spectrum signature of the 2 dyes are very different from each other. Values close to 1 for similarity index indicate that the spectrum signatures are very similar to each other.

### Complexity Index

In the field of numerical analysis, the condition number of a function measures how much the output value of the function can change for a small change in the input argument. The condition number is used to measure how sensitive a function is to changes or errors in the input, and how much error in the output results from an error in the input. A low condition number is said to be well-conditioned, while a high condition number is said to be ill-conditioned.

The condition number is an application of the derivative, and may be defined as the value of the asymptotic worst-case relative change in output for a relative change in input. The condition number is frequently applied to questions in linear algebra, in which case the derivative is straightforward but the error could be in many different directions. The condition number can be computed from the geometry of the matrix.

In the case of multiple fluorochromes (Fluor1 through Fluor M), the complexity index is a condition number of the reference spillover matrix R. While the similarity index is a measure of the one to one interference between two fluorochromes; the complexity index is a measure of the multiple interferences from many fluorochromes to many fluorochromes.

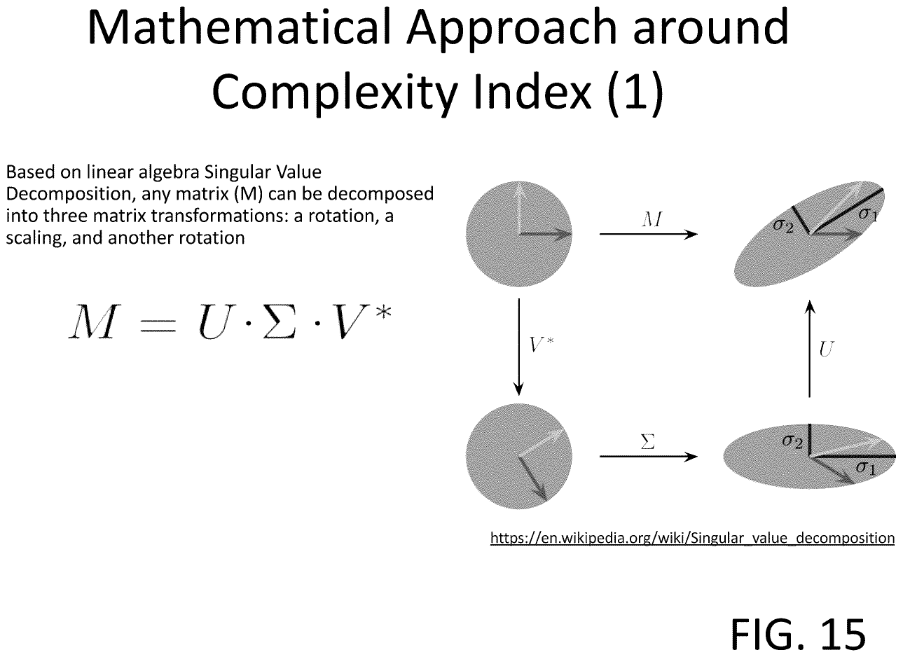

FIG. 15 illustrates a mathematical approach to explain the complexity index. Based on linear algebra and Singular Value Decomposition, any matrix (M) can be decomposed into three matrix transformations: a rotation, a scaling, and another rotation as shown. The matrix M can be represented by three matrices by the following equation: M=U·Σ·V*.

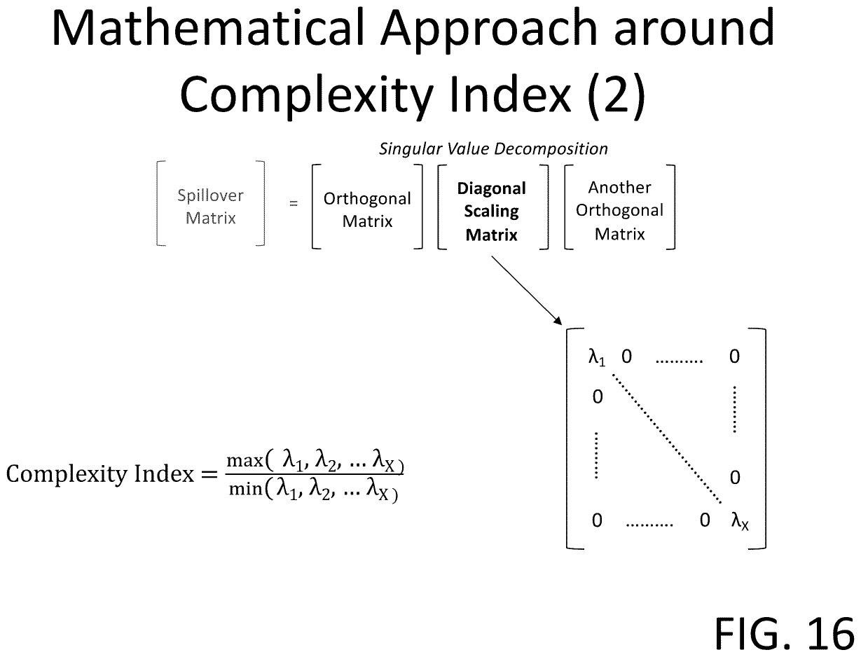

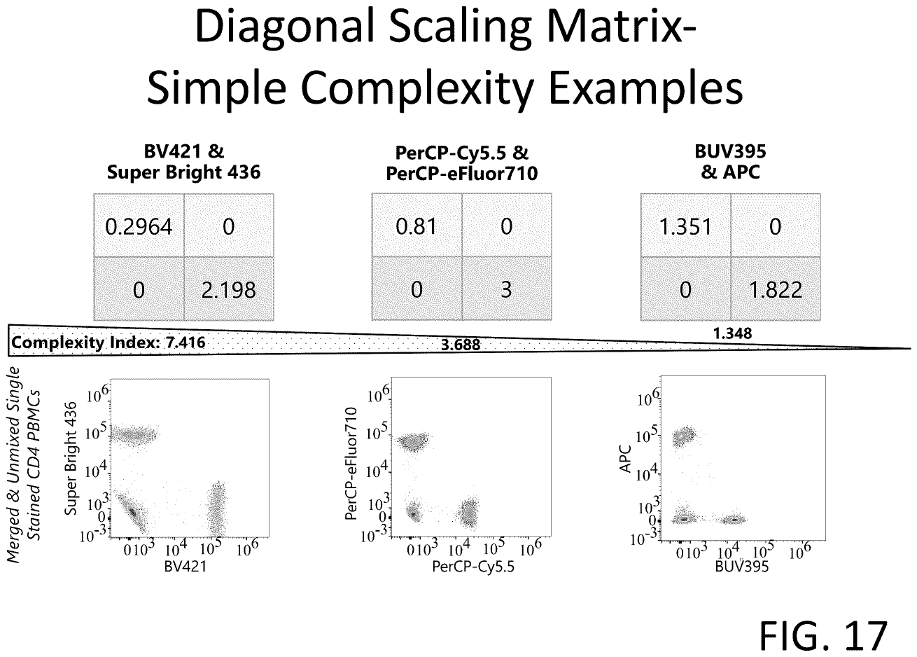

FIG. 16 illustrates the mathematical approach to generating the complexity index. There are different linear algebra theorems that allows decomposition a matrix into several matrix transformations. A single spillover matrix can be represented my three matrixes that when multiplied together generate the original spillover matrix. To generate a complexity index, the similarity matrix is decomposed by using the Singular Value Decomposition theorem in linear algebra. One of the resulting matrixes from that decomposition is a diagonal matrix whose values behave like a scaling factor. The diagonal matrix can be referred to as a diagonal scaling matrix. The magnitude of the diagonal values in the diagonal scaling matrix is directly related to how similar or dissimilar two dyes are to each other. As shown in FIG. 16, one way of computing a complexity index is to choose the maximum value in the diagonal and divide it by the minimum value in the diagonal. Accordingly, one would expect that a larger value for the complexity index is less desirable than a smaller value for the complexity index for a given set of selected fluorochromes that are to be mixed together in a mixed sample.

The complexity index is an overall measure of uniqueness of all dyes in a full spectrum cytometry panel. The lower the value, the easier it will be to work with the dyes in the panel as the overall spread in the panel will be low. The higher the value, the more challenging it will be to work with the dyes in the panel as the overall spread is higher. Well design panels with few dyes (e.g., 10 or less) can have complexity index on the order of values of 2 or 3, for example. Well design larger panels (e.g., 35 to 40 colors or more) will have complexity indexes of around 40 to 50 or less. This general upper value of complexity indexes can be predetermined for each proposed panel and a threshold complexity index can be assigned to a proposed panel. This quantifiable threshold number can be used to compare different panel reagent compositions to select the optimal panel for a given purpose.

FIG. 17 illustrate simple examples of complexity matrices and complexity indexes for pairs of fluorochromes. Example matrices for three different combinations of two dyes. The presence of only one or two large similarity indexes greatly increases the complexity index. The scaling or stretching between two close dyes, shows itself in the data in slanted negative complexity index bar.

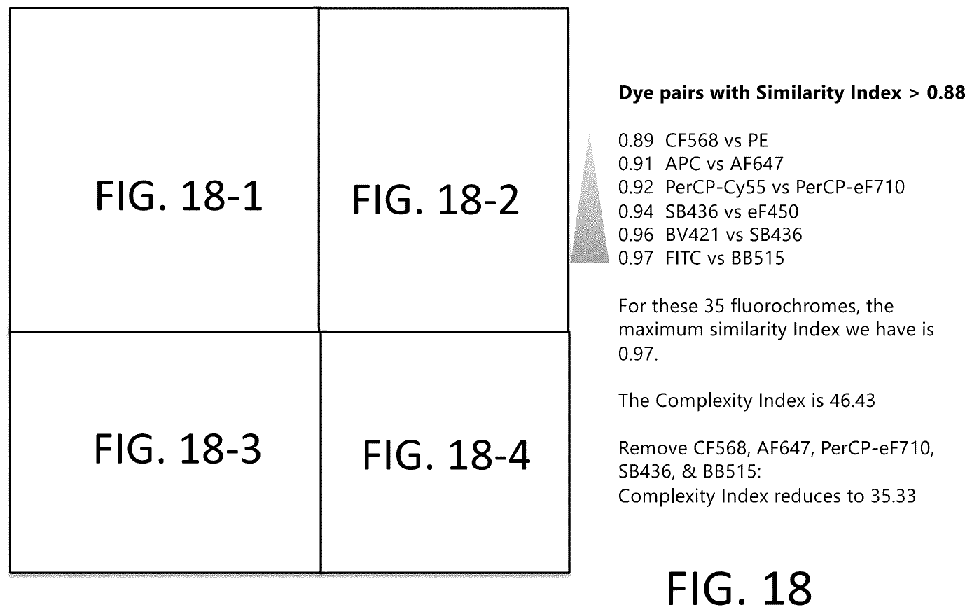

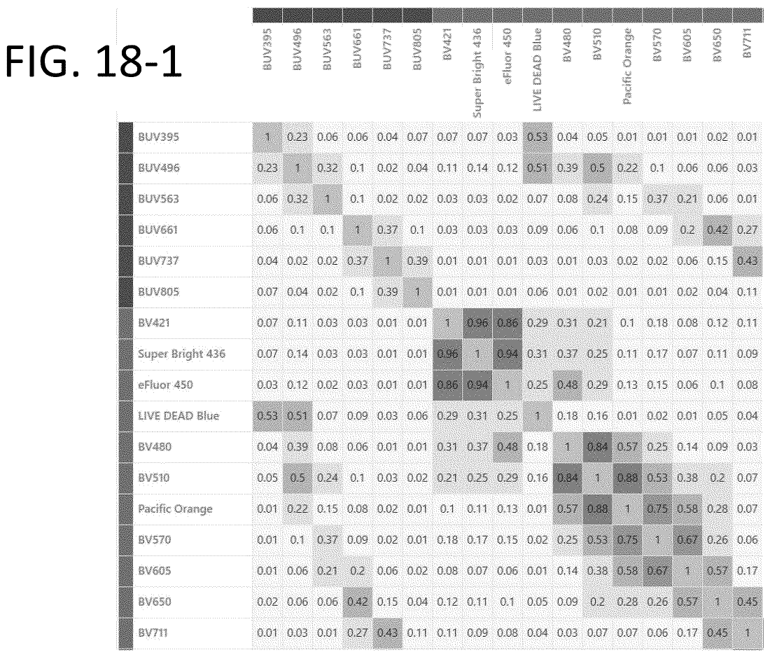

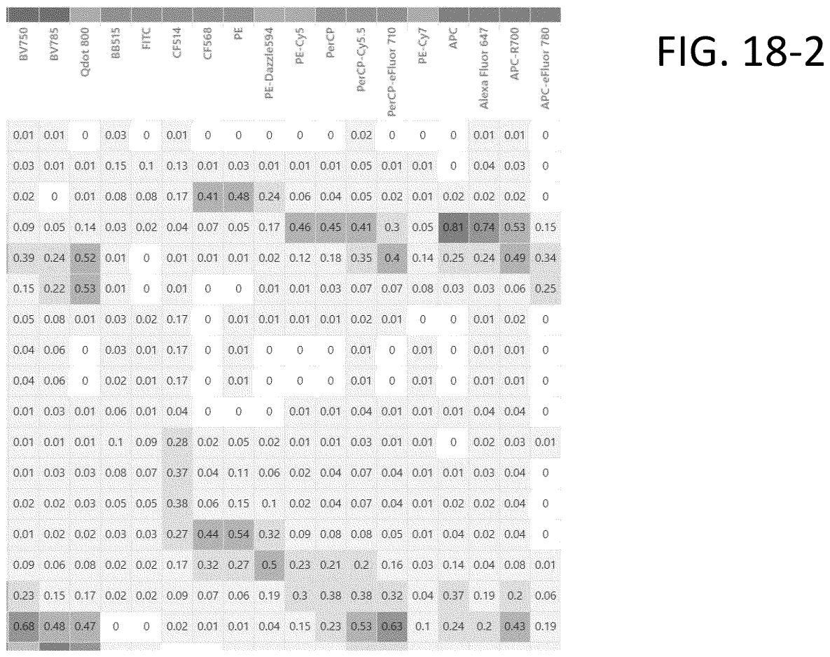

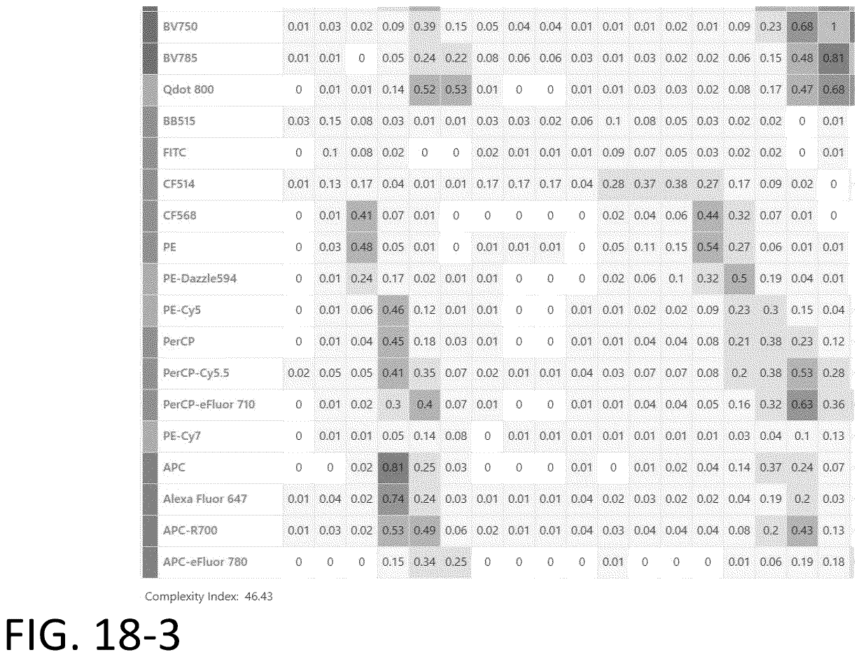

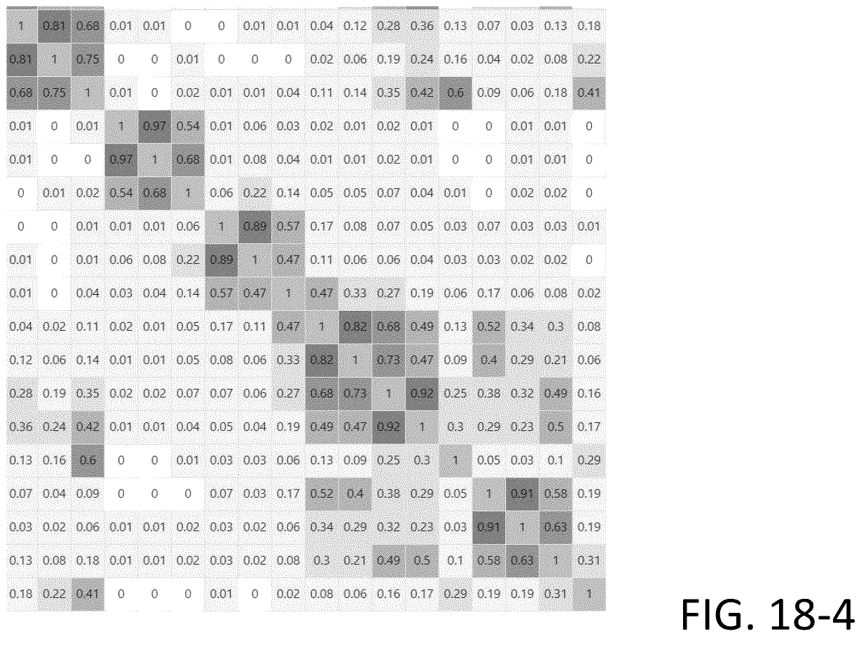

FIG. 18, (separated into FIGS. 18-1 to 18-4, for purposes of reproduction), illustrates a large complexity matrix for analyzing simplicity indexes together and generation of the complexity value. This shows the similarity indices and complexity index for a full 35 color panel including the viability dye. Examples to the right state the complexity index. Identified 35 dyes that are all unique, and have a mixture of brightness levels. In the exemplary similarity and complexity indices of FIG. 18 (FIGS. 18-1 to 18-4), a threshold similarity index of 0.88 was determined. An initial complexity index of 46.53 was determined for this selection of fluorochromes. Six pairs of fluorochromes were found to have a similarity index greater than 0.88. One fluorochrome from the six pairs of fluorochrome were removed from consideration for the panel. In this example, CF568, AF647, PerCP-eF710, SB436, & BB515 were removed (SB436 were in two pairs of fluorochrome with a similarity index greater than 0.88, thus only 5 fluorochromes were removed from consideration. After the removal of the five sub-optimal fluorochromes, the complexity index was calculated again and found to have reduced to 35.33.

The condition number of the reference spillover matrix R is equal to the square root of the condition number of the complexity matrix [R^{T}R].

For a panel of M fluorochromes (Fluor1, Fluor2, . . . , FluorM), the complexity matrix can be determined from the following equation

$$[ {R}^{T} R ] = [ \begin{matrix}{V}_{1 , 1} & {V}_{1 , 2} & … & {V}_{1 , M} \\ {V}_{2 , 1} & {V}_{2 , 2} & … & {V}_{2 , M} \\ ⋮ & ⋮ & ⋮ & ⋮ \\ {V}_{M , 1} & {V}_{M , 2} & … & {V}_{M , M}\end{matrix} ] .$$

The complexity matrix summarizes the mutual similarity of the reference controls provided by the set of fluorochromes used in one flow cytometer run with one biological sample. The Vx,y entries in the complexity matrix are the inner products of the reference controls for two fluorochromes. Thus, the Vx,y entries in the complexity matrix relate to the similarity indices derived from the comparison of two spillover (SOV) vectors of the modeled fluorochromes.

The complexity matrix is derived from the equation [R^{T}R] and the Vx,y values are the elements in the resultant [R^{T}R] matrix, where x and y vary from 1 to M, M being the number of fluorochromes for a given sample/flow cytometry panel. Accordingly, each row in the complexity matrix indicates a different fluorochrome. That is the first row is fluorochrome 1, the second row is fluorochrome 2, and so on and so forth, and the Mth row is fluorochrome M. A row index value (e.g., Fluor1, Fluor2, . . . , FluorM) for each row of the matrix may be used to indicate the selected fluorochrome for a sample. Similarly, each column in the complexity matrix indicates a different fluorochrome.

Note that the complexity matrix is symmetrical, an M by M matrix, where M is the number of fluorochromes. The entries from V1,1 to VM,M along the diagonal of the complexity matrix are expected to be the value of 1 since the same fluorochrome is being compared with itself.

We can take the condition number of the complexity matrix [R^{T}R] representing sensitivity of the matrix to perturbations in value. Then the square root of the condition number of the complexity matrix [R^{T}R] is equal to the condition number of the reference spillover matrix R, that is simply referred to as the complexity index.

### Co-Expression to Simplify Complexity Index Determination

If two fluorochromes co-express on the same stained particle (they interfere with each other), the calculated value of similarity index will provide a measurement of the spillover impact (light emitted/fluorescing at similar wavelengths) between the two fluorochromes. The bigger the similarity index value (closer to one), a more reduced resolution between each is to be expected due to the spillover of these two fluorochromes. The smaller the similarity index value (closer to zero), the greater the resolution (less spillover and spectral overlap) between each of the two fluorochromes

For the panel of M fluorochromes (Fluor1, Fluor2, . . . FluorM), the co-expression of the fluorochromes can be expressed by a symmetrical co-expression matrix as follows:

$$[ \begin{matrix}{CE}_{1 , 1} & {CE}_{1 , 2} & … & {CE}_{1 , M} \\ {CE}_{2 , 1} & {CE}_{2 , 2} & … & {CE}_{2 , M} \\ ⋮ & ⋮ & ⋮ & ⋮ \\ {CE}_{M , 1} & {CE}_{M , 2} & … & {CE}_{M , M}\end{matrix} ] .$$

Each row in the symmetrical co-expression matrix indicates a different fluorochrome. That is the first row is fluorochrome 1, the second row is fluorochrome 2, and so on and so forth, and the Mth row is fluorochrome M. A row index value (e.g., Fluor1, Fluor2, . . . , FluorM) for each row of the matrix may be used to indicate the selected fluorochrome for a sample. The row index value may be used herein to refer to the entire row of values.

If Fluor1 and Fluor2 co-express, then CE_{2,1} and CE_{1,2} are both equal to 1. Otherwise, if Fluor1 and Fluor2 do not co-express, CE_{2,1} and CE_{1,2} are both equal to 0. Therefore, each of the elements in the co-expression matrix is either 1 or 0.

To take out the effects of all the non-co-expressed fluorochromes of the panel, the complexity matrix can be modified by the co-expression matrix using matrix multiplication. The matrix multiplication (or product) of the complexity matrix and the co-expression matrix results in a modified complexity matrix as follows:

$$[ \begin{matrix}{CE}_{1 , 1} {V}_{1 , 1} & {CE}_{1 , 2} {V}_{1 , 2} & … & {CE}_{1 , M} {V}_{1 , M} \\ {CE}_{2 , 1} {V}_{2 , 1} & {CE}_{2 , 2} {V}_{2 , 2} & … & {CE}_{2 , M} {V}_{2 , M} \\ ⋮ & ⋮ & ⋮ & ⋮ \\ {CE}_{M , 1} {V}_{M , 1} & {CE}_{M , 2} {V}_{M , 2} & … & {CE}_{M , M} {V}_{M , M}\end{matrix} ]$$

Each row in the modified co-expression matrix indicates a different fluorochrome. That is the first row is fluorochrome 1, the second row is fluorochrome 2, and so on and so forth, and the Mth row is fluorochrome M. A row index value (e.g., Fluor1, Fluor2, . . . , FluorM) for each row of the matrix may be used to indicate the selected fluorochrome for a sample. The row index value may be used herein to refer to the entire row of values. Similarly, each column in the modified co-expression matrix indicates the different fluorochromes (controls) used in the same sample assay.

Determining the condition number of this modified complexity matrix is a more accurate measurement to use as the complexity index.

Similarly, the co-expression matrix can also be applied to a cross stain index reduction matrix. With a cross stain index reduction matrix modified by the co-expression matrix, a more accurate measurement of cross stain index reduction can be obtained.

There is one more thing we need to consider before assigning cell markers to each fluorochrome. The spread of data clusters needs to be considered.

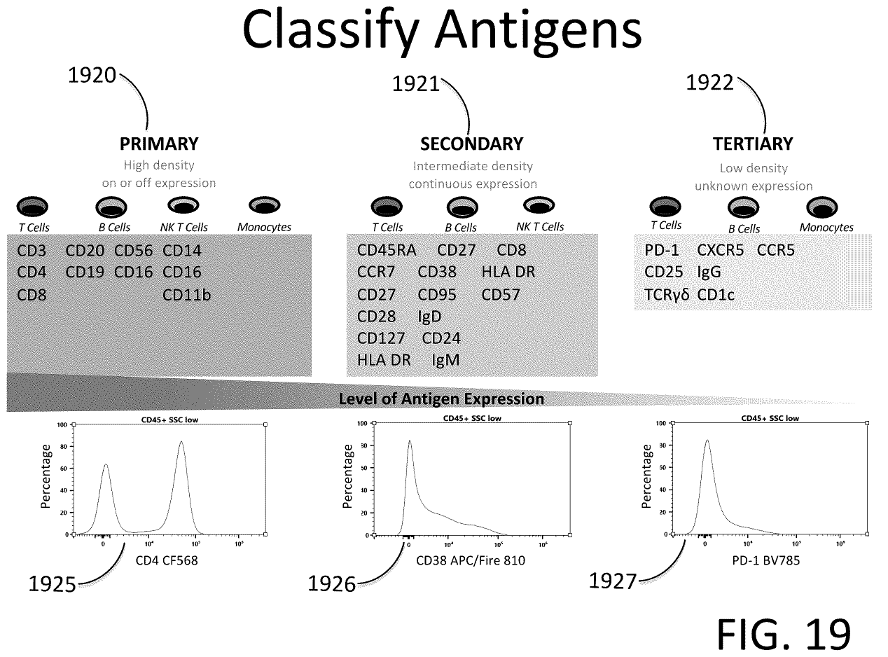

FIG. 19 illustrates the classifications for various antigens. The primary antigens 1921 have a high density on or off expression. The left graph histogram 1925 has very clear bimodal peaks so that positive and negative can be seen the distance between peaks is wide across the spectrum.

The secondary antigens 1921 have an intermediate density with a continuous expression. In the middle graph 1926, there is a continuum between a left peak and a right peak. Some consideration is made to see clearly in the middle between the peaks. A fluorochrome needs to brighter to see over the middle spectrum.

The tertiary antigens 1922 have a low density with an unknown expression. The right graph histogram 1927 does not have a clear separation between the peak and the shoulder peak. Very bright colors need to be used.

The spreading or broadening of peaks can also be an issue when mixing colors together. The data clusters can spread and make it more difficult to detect positive or negatives. A cross stain index values in a cross-stain matrix should also be considered when mixing with other colors.

Accordingly, in panel design, it is desirable to consider the level of antigen expression when selecting fluorochromes to use in a mixed color sample represented by a color flow cytometry panel.

### User Interface for Selecting Fluorochromes.

The design of a flow cytometer can bring flexibility in selecting fluorochromes for labeling biological cells and particles. Full spectrum cytometry has the advantage of detecting the full spectrum signature for each fluorochrome with a full spectrum flow cytometer with at least five lasers and at least 64 detectors. Almost any commercially available fluorochrome can be excited by the lasers of a full spectrum flow cytometer.

With so many options, it is useful to provide a web-based user interface displayable on a monitor or display device to more quickly and more easily choose fluorochromes for use in experiments on biological samples with a full spectrum flow cytometer. A computer or other electronic device, including a processor and input/output devices, is coupled to the internet and the monitor or display device in order to generate and display the web-based user interface. The web-based user interface is generated by a spectrum viewer web-based software tool. The software tool can be executed on a client computer device locally with access to a remote database or remotely on a server computer in communication with the remote database.

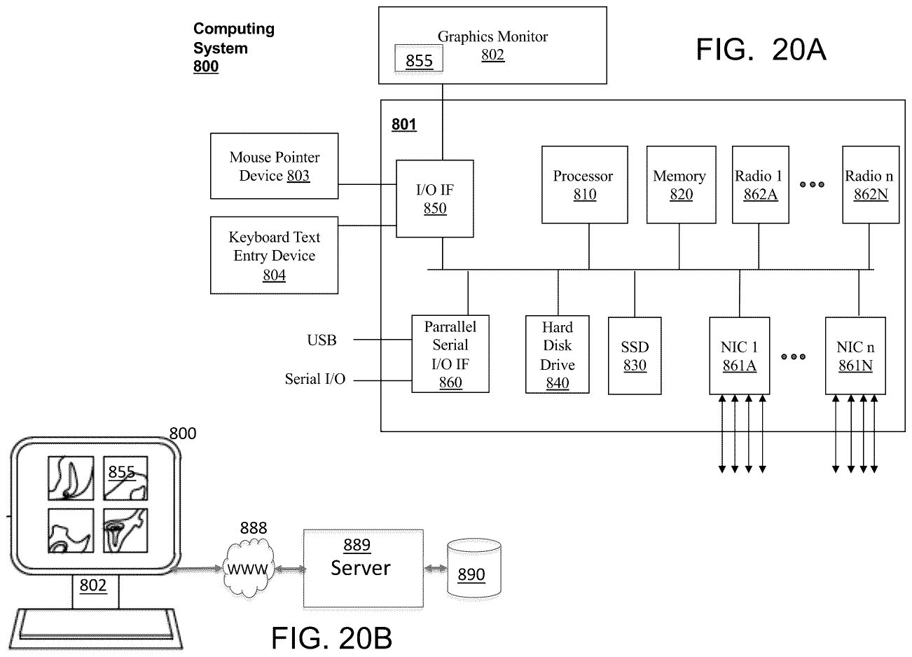

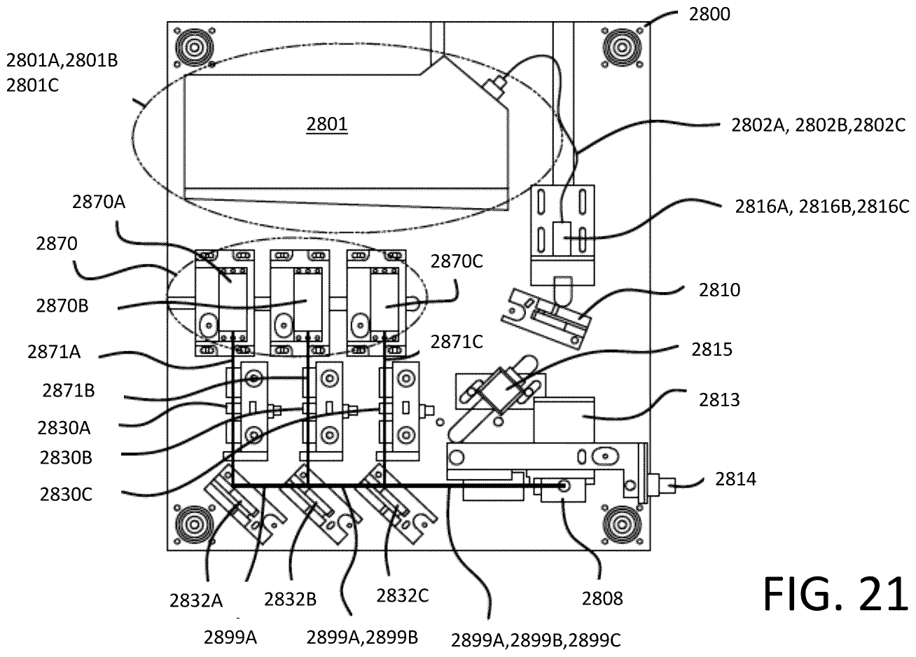

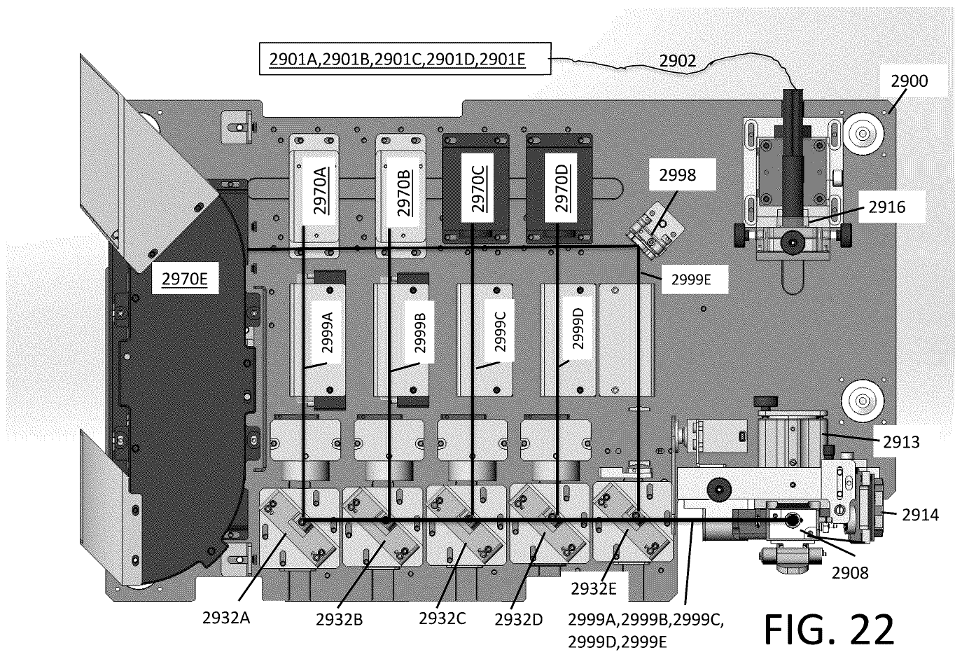

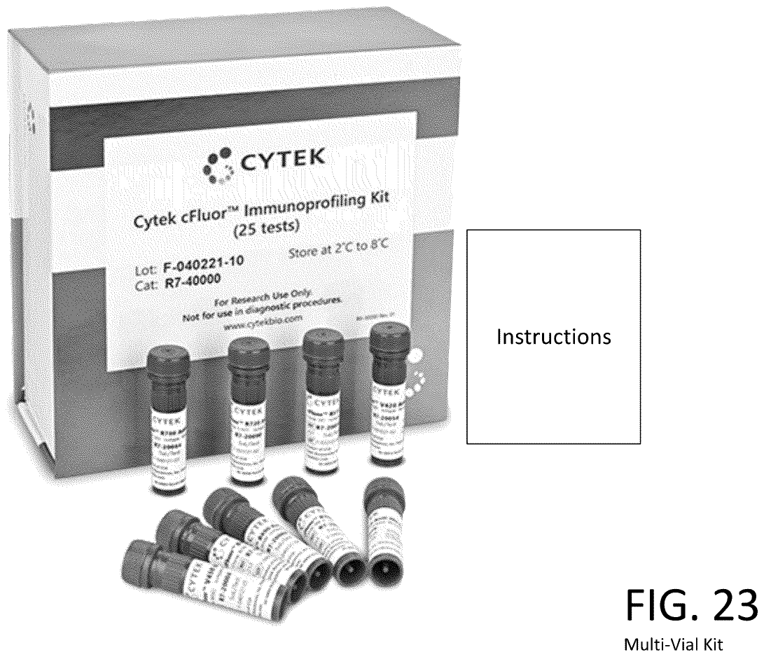

Reference is now made to FIGS. 20A-27C. With the spectrum viewer web-based software tool, users can choose from commercially available fluorochromes that have been previously tested on different configurations of a flow cytometer. For example, FIGS. 22A-22B illustrate an expanding list of fluorochromes tested on a full spectrum cytometer with possible different configuration options.

The spectrum viewer web-based software tool helps users figure out which fluorochromes could be used together on the various configurations of the full spectrum flow cytometer. The software tool can display full spectrum information for over 80 fluorochromes acquired using an assay setting across all of the configurations for the full spectrum flow cytometer.

The spectrum viewer web-based software tool can use the similarity index and the complexity index to further assist a user in selecting fluorochromes than can be used together with the full spectrum flow cytometer in its various configurations.

FIG. 20A a block diagram of a computing system 800 is shown that can execute the software instructions to execute a web browser to graphically display a graphical user interface (GUI) 855 to assist a user in selecting fluorochromes that can be used together with the full spectrum flow cytometer in its various configurations. FIG. 20B is a block diagram illustrating the computing system 800 coupled to a remote computer server 889 over the cloud or internet 888. Monitor 802 illustrates the GUI 855 generated by the server 889 and displayed by the computing system 800. The server 889 is in communication with a database 890 that stores information about the available fluorochromes for use with various configurations of a flow cytometer. The information is determined by running each fluorochrome as a reference sample alone through the flow cytometer. The spillover over vector for each fluorochrome is added into a spillover matrix stored in the database 890. A user can then access the database and select one or more fluorochromes with their underlying data and have graphs charted and the similarity indexes and the complexity index determined.

### Configurable Flow Cytometer.

Referring now to FIGS. 28 and 29, a portion of the optical analysis system of modular flow cytometers are shown. The top view of an optical plate assembly 2800,2900 in a modular configurable flow cytometry system is shown. A modular configurable flow cytometer system is configurable in that different combinations of numbers of lasers (e.g., 1, 2, 3, 4, 5) and numbers of detectors (e.g., 14, 16, 22, 30, 32, 38, 48, 54, 64, 128, 256) can be chosen and installed in the flow cytometer. A flow cytometer can be configured with a combination of one, two three, four, five (5) or more lasers and fourteen, sixteen, twenty-two, thirty, thirty-eight, forty-eight, fifty-four, sixty-four (64) or more detectors. With four or more lasers and forty-eight or more detectors, a flow cytometer can act as a full spectrum flow cytometer capturing more electromagnetic spectra than that of a three laser and a thirty-eight detector configuration.

FIG. 28 shows a top view of an optical plate assembly 2800 for a modular flow cytometry system 100. The optical plate assembly 2800 includes a laser system 2870 having three semiconductor lasers 2870A,2870B,2870C that direct excitation into a flow cell assembly 2808 where a sample fluid flows with sample particles. The laser system 2870 attempts to direct the multiple (e.g., three to five) laser beams in a parallel manner toward the flow cell assembly 2808. The multiple laser beams are slightly offset from one another. The laser system 2870 includes semiconductor lasers 2870A, 2870B, 2870C. The semiconductor laser generate laser beams having different wavelengths, such as 405 nanometers (nm), 488 nm, and 640 nm for example. The output power of the semiconductor lasers can differ as well. For example, a 405 nm semiconductor laser can generate a laser beam that with an output power that is usually larger than 30 milliwatts (mW). The output power of a 488 nm semiconductor laser is usually greater than 20 mW. The output power of a 640 nm semiconductor laser is usually greater than 20 mW. Controller electronics in the flow cytometer control the semiconductor lasers to operate at a near constant temperature and a near constant output power.

An optical system spatially manipulates the optical laser beams 2871A, 2871B, 2871C generated by the semiconductor lasers 2870A, 2870B, 2870C respectively. The optical system includes lenses, prisms, and steering mirrors to focus the optical laser beams onto a fluidic stream carrying biological cells (bio cells). The focused optical laser beam size is typically focused for 50-80 microns (μm) across the flow stream and typically focused for 5-20 μm along the stream flow in the flow cell assembly 2808.

In FIG. 28, the optical system includes beam shapers 2830A-2830C that receive the laser light 2871A, 2871B, 2871C from the semiconductor lasers 2870A-2870C, respectively. The laser light output from the beam shapers 2830A-2830C are coupled into mirrors 2832A-2832C respectively to direct the laser light 2899A, 2899B, 2899C towards and into the flow cell assembly 2808 to target particles (e.g. biological cells) stained with a dye of fluorochromes. The laser light 2899A, 2899B, 2899C is slightly separated from each other but directly substantially in parallel by the mirrors 2832A-2832C into the flow cell assembly 2808.

The laser light beams 2899A, 2899B, 2899C strike the particles/cells as they pass by in the flow stream in the flow cell assembly 2808. The laser light beams 2899A, 2899B, 2899C are then scattered by the particles/cells in the flow stream causing the fluorochromes to fluoresce and generate fluorescent light, and the particles/cells to autoflouresce. A forward scatter diode 2814 gathers on-axis scattered light. A collection lens 2813 gathers the off-axis scattered light and the fluorescent light and directs them together to a dichromatic mirror 2810. The dichromatic mirror 2810 focuses the off-axis scattering light onto a side scatter diode 2815. The dichromatic mirror 2810 focuses the fluorescent light onto at least one fiber head 2816. At least one fiber assembly 2802 routes the fluorescent light toward at least one detector module 2801.

For a more detailed analysis of a biological sample using different fluorescent dyes and lasers wavelengths, multiple fiber heads 2816,2916, multiple fiber assemblies 2802,2902 and multiple detector modules 2801,2901 can be used. For example, three or more fiber heads can be used (e.g., see FIG. 28 with three, and FIG. 29 with five) with three or more detector modules associated with three or more lasers.

FIG. 28 shows three fiber heads 2816A, 2816B, 2816C situated in parallel to receive the fluorescent light and three fiber assemblies 2802A, 2802B, 2802C can be used to direct the fluorescent light to three detector modules 2801A, 2801B, 2801C (only one of which is shown in FIG. 28). The first detector module 2801A is located on the optical plate 2800 while the other detector modules are located on a different level. The three fiber heads 2816A, 2816B, 2816C (and three fiber assemblies 2802A, 2802B, 2802C) for the three different detector modules paired with the three laser light beams 2899A, 2899B, 2899C which are slightly offset from each other (e.g., not precisely co-linear). Accordingly, three fiber heads 2816A, 2816B, 2816C can collect light beam data separately fluorescent light generated by the three laser light beams 2899A, 2899B, 2899C, having three different wavelengths to excite fluorochromes. The three fiber assemblies 2802A, 2802B, 2802C then direct light into three different detector modules (e.g., three different detector modules 2801A, 2801B, 2801C), one of which is located on the optical plate 2800 with others located below the optical plate on a lower level of the flow cytometer.

FIG. 29 shows an optical plate 2900 for a full spectrum flow cytometer having a configuration of five lasers and five detector modules with sixty-four photodetectors. The optical plate 2900 has some similar elements to the optical plate 2800. The optical plate 2900 has five fiber heads 2916 for five detector modules (detector modules located off the optical plate). The optical plate 2900 has five lasers 2970A-2970E, one of which is a violet laser 2970D and another one of which is a UV laser 2970E, for exciting and detecting light over the full visible spectrum, including a portion of the UV wavelength spectrum. The laser light beams 2999A, 2999B, 2999C, 2999D are generated in parallel by the lasers 2970A, 29070B, 29070C, 2970D. The UV laser light beam 2999E is generated by the UV laser 2970E spaced apart and initially perpendicular to the laser beams 2999A, 2999B, 2999C, 2999D. The UV laser light beam 2999E is reflected by a first mirror 2998 on the optical plate and directed to run in parallel to the laser beams 2999A-2999D generated by the respective lasers. The mirrors 2932A, 2932B, 2932C, 2932D, 2932E respectively receive the laser beams 2999A-2999E along their parallel but different paths, and reflect the laser beams to the flow cell assembly 2908 spaced apart in parallel along the same path.

The optical plate 2900 includes a forward scatter detector 2914 that gathers on-axis scattered light from the particles/cells. A collection lens 2913 coupled to the flow cell assembly 2908 gathers the off-axis scattered light, the fluorescent light, the autofluorescent light and directs them together to the fiber heads 2916.

The violet and UV lasers and violet and UV detectors differ from the lasers and detectors of the flow cytometer with the optical plate 2800. The violet and UV detector modules have more photodetectors and therefore detect a wider range of wavelengths of fluorescence light when violet and UV lasers strike a particle/cell. With the UV laser 2970E on the optical plate 2900, the detector modules 2901A, 2901B, 2901C, 2901D, 2901E (collectively referred to as detector modules 2901) are moved off the optical plate 2900. With a plurality of fiber assemblies 2902 and fiber heads 2916, the light from the flow cell 2908 can be directed into the plurality of different detector modules 2901 in different locations of the flow cytometer.

Not only can the excitation be modular (and configurable) in a modular flow cytometry system, but the detection can also be modular. The modular flow cytometry system can also use one or more detector modules 2801,2901 to collect the light beam data. For example, one or more fiber assemblies can direct light from a flow cell into one or more differing detector modules with different arrays of photodetectors and bandpass filters. For full spectrum signatures, a plurality of (four or more) different detector modules can be used. With the selection of detector modules, the total number of photo detectors (e.g., 16, 32, 64, 128) can differ. The differing detector modules may use different numbers of photodetectors to capture light. Generally, the more detectors one has, the more data can be analyzed and the increased spectral resolution can be achieved.

With a spectral flow cytometer, separation of the light beam data in a mixed sample is handled as a data processing operation over the different detector modules and their respective detectors. The data processing operations can be somewhat complex because separation of the light beam data requires more data manipulation (e.g., identifying different wavelengths and separating light beam data accordingly).

Cell geometric characteristics can be categorized though analysis of the forward and side scattering data. The cells in the fluidic flow are labeled by dyes of visible wavelengths ranging from 400 nm to 900 nm or dyes that fluorescent with ultraviolet non-visible wavelengths when excited by an ultraviolet laser. When excited by lasers, the dyes produce fluorescent light, which are collected by the fiber assembly and routed toward a detector module. The modular flow cytometry system maintains a relatively small size, partly with the optical plate assembly using compact semiconductor lasers in the visible spectrum, a multipower collection lens 2813,2913, and compact image detector arrays in the detector modules. That is, the collection lens 2813,2913 contributes to the design of the compact detector modules.

The collection lens can have a short focal length for the its multipower factor (e.g., 11.5× power). The collection lens, an objective lens, has a high numerical aperture (NA) facing the fluorescence emissions to capture more photons in the fluorescence emissions over a wide range of incident angles. The collection lens has a low NA of about facing the fibber head and its collection fiber to launch the fluorescent light into the fiber over a narrow cone angle. Accordingly, the collection lens converts from a high NA on one side to a low NA on the opposite side to support a magnification M in the input channel of each detector module.

The diameter of the core of the collection fiber assembly is between about 400 μm and 800 μm, and the fiber NA is about 0.12 for a core diameter of about 600 μm. The fiber output end can be tapered to a core diameter of between about 100 μm and 300 μm for controlling the imaging size onto the receiving photodiode.

The input end of the collection fiber can also include a lensed fiber end to increase the collection NA for allowing use of a fiber core diameter that is less than about 400 μm. Because the collection fiber has the flexibility to deliver the light anywhere in the flow cytometer system, the use of fiber for fluorescence light collection enables optimization of the location of the receiver assembly and electronics for a compact flow cytometer system.

To manufacture a low-cost flow cytometer, lower cost components can be introduced. An image array in each detector module can be formed out of a solid transparent material to provide a detector module that is reliable, low cost, and compact. Furthermore, the flow cytometer can use low cost off the shelf components, such as thin outline (TO) can photodetectors in the detector modules.

Flow cytometry enables the precise identification (detection) and quantification of immune cell populations, including T cells, B cells, natural killer (NK) cells, dendritic cells, monocytes, macrophages, platelets, and granulocytes. By utilizing preselected combinations of fluorescent-labeled antibodies targeting unique cell markers, researchers can discern and quantify various immune cell subsets within a heterogeneous population. After the fluorescent-labeled antibodies binds to their specific markers, the intensity of fluorescence associated with specific markers form spectroscopic signatures that can be used to evaluate changes in marker expression during immune cell activation, differentiation, or disease progression.

This profiling helps in understanding the functional properties and phenotypic characteristics of immune cell subsets. This is instrumental in diagnosing and monitoring various hematological disorders, such as leukemia and lymphoma. In other embodiments, analyzing specific surface markers or abnormal antigen expression patterns on malignant cells, can assist in subtype classification and disease monitoring to guide appropriate treatment strategies. Researchers of these hematological disorders and treatment strategies can find it advantageous to use prepared kit such as the 24 Color Mouse Immunoprofiling Panel Reagent Kit embodiment of the invention, packaged with preselected markers, clones, and fluorochromes

Flow cytometry assay kits contain specialized reagents designed for flow cytometric cellular analysis. These kits will generally include unique conjugated antibodies and fluorescent dyes or reporter molecules for the detection of target antigens or cells. Referring now to FIG. 35, illustrations of a reagent kit embodiment is disclosed. Each kit can be packaged in shippable containers with handling instructions printed on the box itself. FIG. 35 illustrates an exemplary multi-tube 24 Color Mouse Immunoprofiling Panel Reagent Kit. The tubes of the multi-tube kit can be a test tube or similar non-reactive container for holding small amounts of liquid. The multi-tube can contain the 25 single reference reagents in a plurality of vials (with resealable lids) that are to be mixed by the researcher into a multicolor antibody cocktail in a test tube. The multicolor antibody cocktail can be added into a single tube sample that has biological cells of interest to form a multicolor single tube sample.

Specific instructions can vary by each kit, but in general the following procedure is advantageous for use with the assay kit embodiment of the invention. In certain embodiments, a method is provided for preparing a single-cell suspension suitable for downstream analysis, such as flow cytometry or cell surface marker detection. The method is performed under conditions that preserve cell viability and maintain expression of surface antigens, such as CD62L. In some embodiments, all reagents, incubation steps, and centrifugation steps are carried out at a temperature between approximately 2° C. and 8° C. It has been observed that failure to maintain these temperature conditions may result in reduced or inconsistent detection of CD62L expression.

In one embodiment, the method comprises harvesting a population of cells of interest. All reagents and samples are maintained at a temperature between approximately 2° C. and 8° C. during handling. The harvested cells are passed through a cell strainer or equivalent filtering apparatus to remove aggregates and to obtain a single-cell suspension. In embodiments in which the sample comprises whole blood, the filtering step may be omitted, and the method proceeds directly to the red blood cell lysis step described below.

The cell suspension is centrifuged at approximately 500×g for about 5 minutes at 2-8° C. to form a cell pellet. In instances where red blood cells are present, a red blood cell (RBC) lysis step is performed. If red blood cells are absent, the method may proceed directly to the washing steps described herein.

In one embodiment, a 1×RBC Lysing Buffer is prepared by diluting one part of a 10×RBC Lysing Buffer stock solution with nine parts of cold distilled water, followed by gentle mixing to ensure homogeneity. The cell pellet is then resuspended in approximately 2-3 mL of the cold 1×RBC Lysis Buffer by gentle pipetting. In embodiments utilizing whole blood, the lysis reagent is added directly to the blood sample without a preceding centrifugation step. For example, approximately 1 mL of 1×RBC Lysis Buffer may be added per 100 μL of blood, followed by gentle mixing.

The mixture is incubated for approximately 10-15 minutes at 2-8° C. to permit selective lysis of red blood cells. The sample is subsequently centrifuged at approximately 500×g for about 5 minutes at 2-8° C. The resulting supernatant is carefully decanted, and the remaining cell pellet is resuspended in approximately 5 mL of cold Stain Buffer. The suspension is centrifuged again under similar conditions (500×g for 5 minutes at 2-8° C.) to remove residual lysis buffer.

Following centrifugation, the supernatant is decanted, and the cells are resuspended in phosphate-buffered saline (PBS) at a concentration of approximately 2×10⁶ to 10×10⁶ viable cells per mL. Cell viability may be assessed using trypan blue exclusion or an equivalent viability assay. The resulting single-cell suspension is then suitable for viability staining or other downstream applications.

In certain embodiments, a fixable viability dye solution, such as ViaDye™ Red Fixability Dye, is prepared for use in labeling viable and non-viable cells prior to fixation and analysis, for example, by flow cytometry. The following procedure illustrates one embodiment for preparing and handling the dye reagent to ensure stability and consistent performance.

Prior to reconstitution, the dimethyl sulfoxide (DMSO) used as the solvent is completely thawed to ensure homogeneity. In one embodiment, approximately 100 μL of thawed DMSO is added to a vial containing lyophilized ViaDye™ Red Fixability Dye to yield a 1 mM stock solution. The vial is vortexed or otherwise agitated to achieve complete dissolution of the dye.

The resulting stock solution may be aliquoted into smaller volumes to minimize repeated freeze-thaw cycles and stored at approximately −20° C. until use. Each aliquot is thawed at ambient temperature immediately prior to use and protected from light to prevent photodegradation.

In one embodiment, the stock solution is diluted at a ratio of approximately 1:250 in phosphate-buffered saline (PBS) to prepare a working solution of the viability dye. The working solution may be used at a volume of approximately 5 μL per test or per sample, depending on the total cell number and staining conditions. The prepared reagent is suitable for staining live and dead cells in a single-cell suspension and may be employed in conjunction with fixation and permeabilization protocols for downstream cytometric or imaging-based assays.

In certain embodiments, a viability reference control sample is prepared to provide a standard for distinguishing viable and non-viable cell populations in downstream analytical procedures, such as flow cytometry. The following procedure exemplifies one embodiment for preparing such a control using a fixable viability dye, for example, ViaDye™ Red Fixable Viability Dye.

In one embodiment, a 12×75 mm polystyrene or polypropylene tube is labeled for use as the viability reference control. Approximately 0.3×10⁶ cells are transferred into each tube designated for the single-stain reference or viability control. Cold phosphate-buffered saline (PBS) is added to each tube to bring the total volume to approximately 3 mL, and the suspension is centrifuged at approximately 500×g for about 5 minutes at a temperature of 2-8° C.

Following centrifugation, the supernatant is decanted, and residual liquid is removed by gently blotting the rim of the tube on absorbent paper. The cell pellet is resuspended thoroughly by vortexing to ensure uniform dispersion. Approximately 5 μL of the previously prepared ViaDye™ Red Fixable Viability Dye working solution is added directly to the resuspended cell pellet. The mixture is gently vortexed or otherwise agitated to promote even staining of the cell population.

The sample is incubated for approximately 20 minutes at 2-8° C., protected from light to prevent photobleaching of the fluorescent dye. After incubation, approximately 3 mL of Stain Buffer is added to each tube to quench unbound dye. The suspension is then centrifuged again at approximately 500×g for about 5 minutes at 2-8° C. The supernatant is decanted, and the cell pellet is resuspended thoroughly in Stain Buffer.

In one embodiment, the final cell suspension is adjusted to a volume of approximately 300 μL of Stain Buffer to prepare the sample for data acquisition. If the stained samples are to be stored for longer than approximately two hours prior to analysis, the samples may be fixed in 1% paraformaldehyde at 4° C. according to a fixation procedure described elsewhere herein. If the cells are not fixed, acquisition may be performed within approximately two hours following staining, preferably at a medium flow rate, to ensure accurate viability discrimination.

In certain embodiments, single-color cell reference controls are prepared to enable fluorescence compensation, spectral unmixing, or calibration of multicolor flow cytometry assays. Each reference control corresponds to a single fluorescent marker or dye included in the experimental panel and provides a defined signal reference for that fluorochrome. An unstained control may also be included to establish background fluorescence levels.

In one embodiment, a 12×75 mm polystyrene or polypropylene tube is labeled for each single-stain reference control, including one tube designated as the unstained control. Approximately 0.3×10⁶ cells are added to each tube designated for a reference control. In certain embodiments, the type of cell or reference control used for each marker may be selected according to the recommendations provided in Table 1.

Cold Stain Buffer is added to each tube to bring the total volume to approximately 3 mL, and the suspension is centrifuged at approximately 500×g for about 5 minutes at a temperature of 2-8° C. Following centrifugation, the supernatant is decanted, and residual buffer is removed by gently blotting the tube on an absorbent surface. The cell pellet is mixed thoroughly, for example, by vortexing, to ensure a uniform suspension.

Approximately 1 μL of Fc Shield reagent, or an equivalent Fc receptor-blocking reagent, is added to each tube to minimize nonspecific antibody binding. The suspension is vortexed to mix and incubated for approximately 10 minutes at 2-8° C. without washing. After incubation, the appropriate monoclonal antibody specific to the marker of interest is added directly to the cell pellet. In one embodiment, approximately 1 μL of antibody reagent is used when employing Thermo Fisher Scientific reagents, or approximately 5 μL when employing cFluor® reagents. The suspension is mixed thoroughly to ensure uniform labeling.

The tubes are incubated for approximately 30 minutes at 2-8° C., protected from light to prevent fluorochrome photobleaching. Following staining, approximately 3 mL of cold Stain Buffer is added to each tube, and the cells are centrifuged at approximately 500×g for 5 minutes at 2-8° C. The supernatant is decanted, the rim of the tube is blotted on absorbent paper, and the cell pellet is resuspended thoroughly to ensure homogeneity.

The final cell suspension is adjusted to a volume of approximately 300 μL of Stain Buffer to prepare the sample for data acquisition. If the stained samples are to be stored at 2-8° C. for longer than approximately two hours prior to analysis, the samples may be fixed in 1% paraformaldehyde according to the fixation protocol described elsewhere herein. If the samples are not fixed, data acquisition is preferably performed within approximately two hours following staining, using a medium flow rate setting, to preserve optimal fluorescence signal integrity.

In certain embodiments, single-color bead reference controls are prepared to provide standardized fluorescence references for compensation or spectral unmixing in multicolor flow cytometry assays. Such bead-based controls offer stable and reproducible fluorescence signals and may be used in place of, or in combination with, single-color cell reference controls.

In one embodiment, a 12×75 mm polystyrene or polypropylene tube is labeled for each single-stain reference control, including a tube designated as the unstained control. One drop of Cytek® FSP™ CompBeads, or an equivalent antibody-binding bead reagent, is added to each labeled tube designated for the corresponding fluorochrome or marker. The type of bead reference used for each marker may be selected in accordance with the recommendations provided in Table 1.

Approximately 3 mL of Stain Buffer is then added to each tube. The suspension is centrifuged at approximately 500×g for about 5 minutes to pellet the beads. Following centrifugation, the supernatant is decanted, and residual buffer is removed by gently blotting the rim of the tube on absorbent paper. The bead pellet is mixed thoroughly, for example by vortexing, to ensure a uniform suspension.

The appropriate monoclonal antibody reagent specific to the target fluorochrome is then added directly to the bead suspension. In one embodiment, approximately 1 μL of antibody is added when using Thermo Fisher Scientific reagents, or approximately 5 μL when using cFluor® reagents. The sample is mixed thoroughly to ensure even coating or binding of the antibody to the bead surface.

The mixture is incubated for approximately 15 minutes at room temperature (RT), protected from light to minimize fluorochrome photobleaching. Following incubation, approximately 3 mL of Stain Buffer is added to each tube to remove unbound antibody. The sample is centrifuged again at approximately 500×g for about 5 minutes. The supernatant is decanted, the rim of the tube is gently blotted on absorbent paper, and the bead pellet is thoroughly resuspended to achieve a homogeneous suspension.

The final suspension is adjusted to a total volume of approximately 300 μL of Stain Buffer to prepare the sample for data acquisition. The stained bead reference controls are preferably analyzed within approximately two hours after staining, using a medium flow rate, to ensure optimal fluorescence intensity and consistency across channels.

In certain embodiments, a multicolor-labeled cell sample is prepared for simultaneous detection of multiple cellular markers by flow cytometry. The following procedure illustrates one embodiment for preparing such a multicolor sample, including preparation of an antibody cocktail and sequential staining of cell surface antigens under controlled temperature and light conditions.

In one embodiment, a 12×75 mm polystyrene or polypropylene tube is labeled for each multicolor test sample. An antibody cocktail is prepared immediately prior to use to ensure optimal reagent stability and performance. For each multicolor sample to be processed, the antibody cocktail may include the following components, prepared in the order listed: approximately 10 μL of Brilliant Stain Buffer Plus (added first), 1 μL of Siglec F (CD170) Brilliant Violet™ 711, and 5 μL of each antibody reagent used in the multicolor panel, except for the following reagents, which are added separately at later steps:
cFluor® B515 CD3.
Brilliant Violet™ 480 MertK
cFluor® R780 TCR γδ
cFluor® BYG645 TCR β
cFluor® BYG575 CD64

In one embodiment, Brilliant Stain Buffer Plus is added first to the cocktail tube to minimize fluorochrome interaction. The antibody cocktail may be prepared with an additional volume equivalent to one extra test to compensate for reagent loss during pipetting (for example, preparing sufficient cocktail for six tests when staining five samples). The antibody cocktail is mixed thoroughly and centrifuged at approximately 8,000-10,000×g for about 5 minutes at room temperature to remove antibody aggregates. The prepared antibody cocktail is used immediately and is not stored or reused.

Approximately 0.3-1×10⁶ cells are added to each multicolor sample tube. Cold phosphate-buffered saline (PBS) is added to a total volume of approximately 3 mL, and the suspension is centrifuged at approximately 500×g for about 5 minutes at 2-8° C. The supernatant is decanted, and the rim of the tube is gently blotted on absorbent paper. The cell pellet is resuspended in approximately 100 μL of cold PBS by vortexing or gentle pipetting to achieve a uniform suspension.

Approximately 5 μL of the ViaDye™ Red Fixable Viability Dye working solution is then added to the suspension and mixed thoroughly. The sample is incubated for approximately 20 minutes at 2-8° C., protected from light. Following incubation, approximately 3 mL of cold Stain Buffer is added, and the washing step (centrifugation at 500×g for 5 minutes at 2-8° C., followed by decanting and blotting) is repeated.

The cell pellet is resuspended in approximately 100 μL of cold Stain Buffer. Approximately 1 μL of Fc Shield reagent is added to block Fc receptor binding, and the mixture is vortexed to mix thoroughly. The suspension is incubated for approximately 10 minutes at 2-8° C. without washing.

Subsequently, approximately 5 μL of cFluor® B515 CD3 antibody and 1 L of MertK Brilliant Violet™ 480 antibody are added to the suspension, followed by gentle mixing by pipetting or vortexing. The mixture is incubated for approximately 10 minutes at 2-8° C., protected from light, without washing. Next, approximately 5 μL each of cFluor® R780 TCR γδ, cFluor® BYG645 TCR β, and cFluor® BYG575 CD64 antibodies are added, and the sample is mixed thoroughly. The mixture is again incubated for approximately 10 minutes at 2-8° C., protected from light, without washing.

Following sequential addition of these reagents, approximately 91 μL of the previously prepared antibody cocktail is added to the sample, followed by thorough mixing. The sample is incubated for approximately 30 minutes at 2-8° C., protected from light, with gentle mixing once more at approximately 15 minutes to maintain suspension uniformity.

After incubation, approximately 3 mL of cold Stain Buffer is added to each tube, and the samples are centrifuged at approximately 500×g for about 5 minutes at 2-8° C. The supernatant is decanted, and the tube is gently blotted on absorbent paper. The final cell pellet is resuspended in approximately 400 μL of Stain Buffer to prepare the sample for data acquisition.

In embodiments where the stained samples require storage at 2-8° C. for more than approximately two hours before analysis, the cells may be fixed in 1% paraformaldehyde according to the fixation method described herein. If fixation is not performed, the multicolor samples are preferably analyzed within approximately two hours following staining, using a medium flow rate setting to maintain fluorescence signal stability and resolution across all detection channels.

Fluorochromes or dyes disclosed have a peak emission and the identity of the disclosed fluorochromes can be related to that peak emission. For example, cFluor V420 has a peak emission of 420 nm and cFluor V547 has a peak emission of 547 nm. It should be understood that the invention is not limited to the specific identity of the fluorochrome disclosed in the 24-color mouse immunoprofiling panel. Other similar fluorochromes can be substituted and will still be within the scope of the embodiments. For example, cFluor V420 can be substituted for Super Bright 436. A similar fluorochrome with a peak emission within ±30 nm of the peak emission of the disclosed fluorochromes would still be considered within the scope of the claims.

Some portions of the preceding detailed description have been presented in terms of algorithms and symbolic representations of operations on data bits within a computer memory. These algorithmic descriptions and representations are the tools used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be kept in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The embodiments are thus described. While certain exemplary embodiments have been described and shown in the accompanying drawings, it is to be understood that such embodiments are merely illustrative of and not restrictive on the broad invention, and that the embodiments not be limited to the specific constructions and arrangements shown and described, since various other modifications may occur to those ordinarily skilled in the art.

When implemented in software, the elements of the embodiments of the invention are essentially the code segments to perform the necessary tasks. The program or code segments can be stored in a processor readable medium for execution by a computer processor. The “processor readable medium” may include any medium that can store information. Examples of the processor readable medium include an electronic circuit, a semiconductor memory device, a read only memory (ROM), a flash memory, an erasable programmable read only memory (EPROM), a floppy diskette, a CD-ROM, an optical disk, a hard disk, a fiber optic medium, a radio frequency (RF) link, etc. The computer data signal may include any signal that can propagate over a transmission medium such as electronic network channels, optical fibers, air, electromagnetic, RF links, etc. The code segments may be downloaded using a computer data signal via computer networks such as the Internet, Intranet, etc, and stored in a storage device (processor readable medium).

While this specification includes many specifics, these should not be construed as limitations on the scope of the disclosure or of what may be claimed, but rather as descriptions of features specific to particular implementations of the disclosure. Certain features that are described in this specification in the context of separate implementations may also be implemented in combination in a single implementation. Conversely, various features that are described in the context of a single implementation may also be implemented in multiple implementations, separately or in sub-combination. Moreover, although features may be described above as acting in certain combinations and even initially claimed as such, one or more features from a claimed combination may in some cases be excised from the combination, and the claimed combination may be directed to a sub-combination or variations of a sub-combination. Accordingly, while embodiments of the invention have been particularly described, they should not be construed as limited by such embodiments, but rather construed according to claims that follow below.

What is claimed is:

1. A twenty-four (24) color mouse immunoprofiling panel using a spectral flow cytometer, the panel comprising:
VIOLET

| SPECIFICITY/MARKER | ANTIBODY/CLONES | FLUOROCHROME |
|---|---|---|
| XCR1 | ZET | cFluor V435 |
| CD11c | N418 | cFluor V450 |
| MertK | DS5MMER | BV480 |
| NK1.1 | PK136 | cFluor V505 |
| CD45 | 30-F11 | cFluor V547 |
| CD8 | 53-6.7 | cFluor V605 |
| B220/CD45R | RA3-6B2 | cFluor V670 |
| Siglec F (CD170) | 1RNM44N | BV711 |
| CD4 | RM4-5 | cFluor V780 |

BLUE

| SPECIFICITY/MARKER | ANTIBODY/CLONES | FLUOROCHROME |
|---|---|---|
| CD3 | 17A2 | cFluor B515 |
| CD19 | 1D3 | cFluor B532 |
| Ly-6G | 1A8 | cFluor B548 |
| CD64 | X54-5/7.1 | cFluor BYG575 |
| F4/80 | BM8.1 | cFluor BYG610 |
| TCR beta | H57-597 | cFluor BYG645 |
| CD317 | 927 | cFluor BYG667 |
| CD49b | HMa2 | cFluor BYG710 |
| I-A/I-E | M5/114.15.2 | cFluor BYG750 |
| CD62L | MEL-14 | cFluor BYG781 |

RED

| SPECIFICITY/MARKER | ANTIBODY/CLONES | FLUOROCHROME |
|---|---|---|
| CD25 | PC61.5 | cFluor R659 |
| CD11b | M1/70 | cFluor R685 |
| CD44 | IM7 | cFluor R720 |
| | | ViaDye Red |
| TCR g/d | GL3 | cFluor R780 |
| Ly-6C | HK1.4 | cFluor R840. |

2. A method of a twenty-four (24) color flow cytometry panel using a spectral flow cytometer, the method comprising:
selecting twenty-four (24) cell markers for biological cells of interest as recited in claim 1;
selecting twenty-four (24) clones as recited in claim 1 specific to the twenty-four (24) cell markers;
identifying twenty-four (24) fluorochromes recited in claim 1, to be used in the twenty-four (24) color flow cytometry panel;
conjugating the twenty-four (24) clones with the twenty-four (24) fluorochromes to form twenty-four (24) fluorochrome conjugated antibodies;
calibrating at least three (3) lasers and at least twenty-five (25) photodetectors in the spectral flow cytometer;
comparing resolution of each fluorochrome in a multicolor single tube sample versus a twenty-four (24) combined control sample;
staining the biological cells of interest with the twenty-four (24) fluorochrome conjugated antibodies, comprising the twenty-four (24) fluorochromes and twenty-four (24) clones specific to the twenty-four (24) cell markers, to create a multicolor single tube sample;
analyzing the multicolor single tube sample through the spectral flow cytometer including the at least twenty-five (25) photodetectors;
receiving data from the at least twenty-five (25) photodetectors of the spectral flow cytometer; and
processing the received data using a computer processor to analyze the multicolor single tube sample of the biological cells of interest.

3. The method of claim 2, wherein pairing the twenty-four (24) fluorochromes with the twenty-four (24) selected cell markers comprises;
assigning a dimmest fluorochrome to a highest expressing antigen;
assigning tertiary markers to bright fluorochromes; and
avoiding placing highly expressed antigens adjacent to co-expressed antigens with lower expression for fluorochromes with a same primary excitation laser or similar emission wavelengths.

4. The method of claim 2, wherein the biological cells of interest comprise splenocyte and bone marrow cells.

5. The method of claim 2, wherein selecting the twenty-four (24) fluorochromes comprises, quantifying uniqueness of each of the twenty-four (24) fluorochromes.

6. The method of claim 4, wherein selecting the twenty-four (24) fluorochromes comprises, analyzing spectra of each of the twenty-four (24) fluorochromes using the spectral flow cytometer.

7. The method of claim 6, wherein selecting the twenty-four (24) fluorochromes comprises, comparing the spectra of a pairing of twenty-four (24) or fluorochromes; and assigning a similarity index to each pairing of fluorochromes.

8. The method of claim 7, wherein selecting the twenty-four (24) fluorochromes further comprises, determining a threshold similarity index value and not selecting at least one fluorochrome of the pair of fluorochromes with a similarity index value higher than the threshold similarity index value.

9. The method of claim 8, wherein selecting the twenty-four (24) fluorochromes comprises, choosing the twenty-four (24) fluorochromes with a lowest similarity index value.

10. The method of claim 9, wherein a range for the lowest-similarity index value that will produce high resolution data is between 0.95 and 1.0.

11. The method of claim 2, wherein identifying the twenty-four (24) fluorochromes comprises:
determining a complexity index for the twenty-four (24) fluorochromes; and
determining a threshold complexity index above which the twenty-four (24) fluorochromes are not considered optimal.

12. The method of claim 11, wherein a range of the threshold complexity index is between fifty-two (52) to fifty-six (56).

13. A reagent kit for mouse cell immunoprofiling by a spectral flow cytometer having at least three (3) lasers, the reagent kit comprising:
a plurality of test tubes having one or more reagents of a reagent composition of fluorochromes and conjugated antibodies specific to cell markers as recited in claim 1.

14. The reagent kit of claim 13, wherein the at least three (3) lasers are violet, red, and blue lasers.

15. A method of gating a twenty-four (24) color mouse cell immunoprofiling panel, the method comprising;
sequentially gating for total live splenocytes by red blood cell exclusion, singlets, and viable CD45+ events;
excluding debris from the viable CD45+ events;
gating for NK cells and CD3+ NKT-like cells by expression of CD49b versus NK1.1, and subsequently gated on CD3 versus B220;
identifying basophils (CD49b+CD45dim) after excluding the 49b+NK1.1+;
determining plasmacytoid dendritic cells (PDCs, CD317++B220+) after excluding non-basophils;
identifying B cells in non-PDC cells by positive expression of CD19 when gated versus B220;
identifying γδT cells by co-expression of CD3 and TCRγδ from B220−CD19− gated event;
gating for T cells on a CD3 versus TCRβ plot (gating on CD3+TCRβ+), from the non-γδ-T events;
distinguishing CD4+ and CD8+ T cell subsets from the total T cell gate (CD4+CD8− and CD8+CD4− events) and then further subdivided into naïve and memory subsets by expression of CD62L and CD44 for each T cell subset (CD4+ and CD8+);
identifying CD4+CD25+ T cells from a CD4 versus CD25 plot; and
gating for dendritic cells (DCs) on a CD11c versus I-A/I-E plot from non-T cell events and DCs are further subdivided into cDC1 and cDC2 populations using CD8 vs. XCR1 and CD4 vs. CD11b plot.
